(19) United States
(12) Patent Application Publication
(10) Pub. No.: US 2024/0418695 A1
(43) Pub. Date: Dec. 19, 2024

(54) SYSTEM AND METHOD FOR DETECTING AND ANALYZING THE PHYSIOLOGICAL CONDITION AND HEALTH OF PLANTS

(71) Applicant: Soli Organic, Inc., Rockingham, VA (US)

(72) Inventors: Tessa Pocock, Laramie, WY (US); Kenneth Brownlee, Palo Alto, CA (US); Ingo Speier, Saanichton (CA)

(21) Appl. No.: 18/744,066

(22) Filed: Jun. 14, 2024

Related U.S. Application Data
(60) Provisional application No. 63/508,051, filed on Jun. 14, 2023.

(51) Int. Cl. G01N 33/00; G01N 21/01

(52) U.S. Cl. CPC G01N 33/0098; G01N 21/01; G01N 2021/0125

(57) ABSTRACT

A system, devices, and methods for remotely monitoring biological and physiological characteristics of plants is provided. More specifically, applying an excitation light source with a relatively short wavelength to a plant, using a detection apparatus to detect fluorescence emitted from the plant in response to the excitation light, and recording the data corresponding to such emitted fluorescence. Such recorded data can be analyzed to determine the general health condition and various characteristics of the plant in real time.

### CROSS REFERENCE TO RELATED APPLICATIONS

This application claims priority to U.S. provisional application No. 63/508,051, filed Jun. 14, 2023, the entirety of which is hereby incorporated herein by reference for all purposes.

### FIELD

The present disclosure generally relates to remotely monitoring biological and physiological characteristics of plants. More specifically, the present disclosure relates to applying an excitation light with a relatively short wavelength to a plant, detecting a fluorescence response of the plant in response to the excitation light, and recording the data corresponding to the detected fluorescence response.

### BACKGROUND

The needs and requirements of the agricultural industry continuously change over time, and the agricultural industry continuously evolves to meet these changing needs and requirements. This is especially true for vertical farms under sole source lighting as the growing systems are physically difficult to reach and need to be monitored using some type of remote sensing. As the world's population continues to grow, requirements for food to feed the world's population proportionally increase, while the availability and cost of arable land and resources such as fertilizer and water decreases. To meet these challenges, the agricultural industry needs to turn to innovation and technology to provide methods and systems that significantly increase food yields while using less resources.

Generally, the use of certain technologies to increase the yield of food production under cover is referred to as controlled environment agriculture (CEA). CEA applies to both greenhouses and vertical farms where it is critical to have fine control of both the environment and the physiological and biological needs of the plants.

One method of detecting the biological state of a plant is to detect light emitted by a plant's chlorophyll molecules, a phenomenon known as chlorophyll a fluorescence. When a plant is illuminated with a light source, the electrons in the chlorophyll molecule are excited from lower to higher energy states. As the chlorophyll molecules return to ground state (i.e., non-excited state), the plant's chlorophyll molecules emit chlorophyll fluorescence in the red and far-red regions of the spectrum. The detection, recording, and subsequent analysis of this chlorophyll fluorescence is useful in determining the physiological state of the plant. In one approach, devices for measuring photosynthesis and photochemical activity through measurement of chlorophyll a fluorescence are either in direct contact to the plant leaf or up to a maximum of 2-3 cm from a plant leaf. The field of view of such devices is usually limited from a small point to 1 cm.

A common system includes a blue or white light excitation source that is directed to a plant leaf where it is absorbed by pigments including chlorophyll a in the plant, a detector that measures chlorophyll a fluorescence emitted by the plant in the red range (around 685 nm) and/or the far-red (720-750 nm) range, and a control system to control the excitation source and detector to manage background noise and evaluate reaction kinetics. However, such systems provide limited crop analysis and capabilities.

Upon excitation with short wavelength light, the plant may also emit blue/green fluorescence in the blue or green range of light (approximately 450 nm-550 nm). Blue and green fluorescence itself is not typically used for stress detection but it is reported that on leaf or imaging fluorescence sensing is able to detect biotic and abiotic stresses as well as relative amounts of hydroxycinnamic acids in cell walls (Morales et al. 1994, Ortiz-Bustos et al. 2017, Hideg et al. 2002, Buschmann et al. 2000).

### SUMMARY

Disclosed herein are novel systems and devices for monitoring plant status of subject plants (e.g., health, physiological condition, stress, etc.) and novel methods for analyzing measurement data to determine the plant status of subject plants. Once the status of the plant is determined, the system, device, and methods can be used to adjust environmental conditions, to increase or maintain the yields and favorable characteristics of the plant. The novel systems and methods disclosed herein go well beyond the prior art systems and methods, which detect only narrow bandwidths of red or far-red light. The systems and methods disclosed herein detect a significantly broader range of fluorescence emitted from plants remotely to determine the health, stress, and physiological activity of the plant.

Generally stated, the systems and methods disclosed herein (i) illuminate a target plant with a relatively shortwave light source; (ii) detect and record the resulting fluorescence emitted from the plant in real time,; (iii) use such information to assess the condition and characteristics of the plant; in an additional step the system may communicate with other components of the CEA systems and (iv) timely adjust environmental parameters including light based on such analysis to achieve the highest possible yield and favorable characteristics of the plant. The systems and methods can be utilized for a variety of operations, including, but not limited to, detecting stress in the plant, detecting levels of phenolic compounds or other chemicals present in the plant, and detecting physiological activity in the plant. It will be understood that a plant's “stress” can be indicative of a variety of conditions that require mediation such as lack of appropriate nutrients, drought, disease and insect pressure, temperature changes, and other such conditions.

### BRIEF DESCRIPTION OF THE DRAWINGS

FIG. 1 is a schematic diagram depicting an example measurement system for monitoring plant status by measuring fluorescence response of a subject plant within an environment.

FIG. 2 is a schematic diagram depicting an example computing system that includes the measurement system of FIG. 1.

FIGS. 3A and 3B are flow diagrams depicting example methods for monitoring plant status.

FIG. 4 depicts a timeline of an example measurement operation performed by the measurement system of FIGS. 1 and 2.

FIG. 5 depicts a timeline of another example measurement operation performed by the measurement system of FIGS. 1 and 2.

FIG. 6 depicts a timeline of another example measurement operation performed by the measurement system of FIGS. 1 and 2.

FIG. 7 is a graph showing the average fluorescence spectra for basil (Ocimum basilicum) plants grown under controlled conditions with adequate irrigation.

FIG. 8 is a graph showing the average fluorescence spectra for basil (Ocimum basilicum) plants grown under controlled conditions without irrigation and under drought conditions.

FIG. 9 is a graph showing average ratios of different wavelengths of detected fluorescence over time for a number of control basil plants.

FIG. 10 is a graph showing average ratios of different wavelengths of detected fluorescence over time for a number of stressed plants subjected to drought.

FIG. 11 is a schematic diagram depicting an example detection module.

FIG. 12 is a schematic diagram depicting an example emitter module, which is also referred to as an excitation module.

FIG. 13 is a schematic diagram depicting additional aspects of a controller of the measurement system of FIG. 1.

FIG. 14 is a schematic diagram depicting an example computing system of which the computing system of FIG. 2 is an example.

### DETAILED DESCRIPTION

As introduced above, there is a need in the agricultural and controlled environment industries for the ability to remotely sense significantly more and relevant information from plants, analyze such information, and appropriately adjust any environmental conditions or factors required to maximize the desired characteristics of plants.

The present disclosure deals with a novel plant monitoring system to monitor plant health remotely and non-invasively in real time. Such information can be used to provide manual or automatic control of one or more environmental parameters necessary to achieve the highest possible yield from the crop.

In at least some examples, the emitted fluorescence from plants can be detected and recorded across a broad spectrum of light ranging from 400 nanometers (nm) to 780 nm, which includes blue (400-500 nm), green (500-600 nm), red (600-700 nm), and far-red light (700-780 nm). Such recorded data can be analyzed using novel methods to determine the plant status, including health, stress, and physiological conditions and various characteristics of the plant in real time.

A system, methods, and various devices for monitoring plant status are disclosed. An example method includes performing a plurality of measurement operations periodically over a period of time. Each measurement operation includes: emitting, via one or more emitters, excitation light within an emission wavelength range into an environment containing one or more subject plants, prior to emitting the excitation light, obtaining via one or more detectors having a field of view containing the one or more subject plants, at least a first measurement of light intensity within a first observation wavelength range that differs from the emission wavelength range, and at least a first measurement of light intensity within a second observation wavelength range that differs from the emission wavelength range and the first observation wavelength range, and during and/or after emission of the excitation light, obtaining via the one or more detectors, at least a second measurement of light intensity within the first observation wavelength range, and at least a second measurement of light intensity within the second observation wavelength range.

For each measurement operation performed, the method further includes subtracting the first measurement of light intensity from the second measurement of light intensity within the first observation wavelength range to obtain a first measurement of fluorescence response of the one or more plants within the first observation wavelength range.

For each measurement operation performed, the method further includes subtracting the first measurement of light intensity from the second measurement of light intensity within the second observation wavelength range to obtain a second measurement of fluorescence response of the one or more plants within the second observation wavelength range.

For each measurement operation performed, the method further includes, comparing the first measurement of fluorescence response to the second measurement of fluorescence response to obtain a result indicative of the plant status, and outputting the result indicative of the plant status for each measurement operation.

In at least some examples, the method can further include outputting an alert responsive to any of the results exceeding a threshold value.

In at least some examples, the method can further include outputting a control signal to one or more physical actuators to control an operating condition of the environment containing the one or more plants responsive to any of the results exceeding a threshold value.

In at least some examples, the method can further include identifying a rate of change of the results over the period of time, comparing the rate of change to a threshold rate of change, and outputting an alert responsive to the rate of change exceeding the threshold rate of change.

In at least some examples, the method can further include identifying a rate of change of the results over the period of time; comparing the rate of change to a threshold rate of change; and outputting a control signal to one or more physical actuators to control an operating condition of the environment containing the one or more plants responsive to the rate of change exceeding the threshold rate of change.

In at least some examples, the emission wavelength range is less than the first observation wavelength range and the second observation wavelength range. As an example, the emission wavelength range includes ultraviolet light, the first observation wavelength range includes red light and/or near red light, and the second observation wavelength range includes green light and/or blue light.

In at least some examples, the first measurement of light intensity within the first observation wavelength range and the first measurement of light intensity within the second observation wavelength range are captured in parallel via the one or more detectors during the same sample period; and the second measurement of light intensity within the first observation wavelength range and the second measurement of light intensity within the second observation wavelength range are captured in parallel via the one or more detectors during the same sample period.

In at least some examples, the first measurement of light intensity within the first observation wavelength range and the first measurement of light intensity within the second observation wavelength range are captured in sequence via the one or more detectors during different sample periods; and the second measurement of light intensity within the first observation wavelength range and the second measurement of light intensity within the second observation wavelength range are captured in sequence via the one or more detectors during different sample periods.

The preceding example method can be performed in whole or in part by an individual device, such as a controller of the measurement system disclosed herein, or a combination of devices that form a system, as examples.

Additionally, disclosed herein are novel systems, devices, and methods for remotely sensing a fluorescence response from plants and analyzing measurement data to determine the health and characteristics of the plant. Once the health of the plant is determined, the system and methods can adjust environmental conditions, such as light, water, nutrients, etc. as required to increase or maintain the yields and favorable characteristics of the plant. The novel systems and methods disclosed herein go well beyond the prior art systems and methods, which detect only narrow bandwidths of red or far-red light. The systems and methods disclosed herein are capable of detecting a significantly broader range of fluorescence emitted from plants remotely to determine the health, stress, and physiological activity of the plant.

Generally stated, the systems and methods disclosed herein (i) illuminate a target plant with a relatively shortwave light source; (ii) detect and record the resulting fluorescence emitted from the plant in real time,; (iii) use such information to assess the condition and characteristics of the plant; and in an additional step the system may communicate with other components of the CEA systems and (iv) timely adjust environmental parameters including light based on such analysis to increase or achieve the highest possible yield and favorable characteristics of the plant. The systems and methods can be utilized for a variety of operations, including, but not limited to, detecting stress in the plant, detecting levels of phenolic compounds or other chemicals present in the plant, and detecting physiological activity in the plant. It will be understood that a plant's “stress” can be indicative of a variety of conditions that require mediation such as lack of appropriate nutrients, drought, disease and insect pressure, temperature changes, and other such conditions.

In one embodiment, an exemplary system includes an LED excitation source at the wavelength of 365 nm and a detector arranged to detect the resulting fluorescence across a range of 450 nm to 780 nm. The system further includes a measurement system containing a controller configured to i) send signals to a emission module to control the excitation light and amplitude ii) send signals to a detector module to control the detection of fluorescence and iii) receive the measured fluorescence signals from the detection module. The controller may also store the resulting data that corresponds to the properties and levels of the detected fluorescence to remote and/or on-premises data storage, and perform some preliminary analysis or processing of measurement data. A software application on a computer, tablet or phone may further analyze the data to determine the current condition and characteristics of the plant and provide a time resolved view of the plant to the user (e.g., as a graph or other user interface). Optionally, the software application can be arranged to communicate with equipment capable of modifying the growing environment of the plant. For example, the software application may communicate with equipment that controls the amount of light, irrigation, temperature, and relative humidity of the ambient air adjacent to the plant.

In at least some examples, the disclosed system does not rely on highly complex equipment and can be constructed from relatively cost-effective components such as the LED light source described above and photodiode detectors that are arranged to detect Blue/Green fluorescence (between 450 nm and 550 nm) and R/FR chlorophyll a fluorescence (between 675 nm 750 nm) and microcontrollers. While the system is described as using LEDs as a light source, other light sources, such as lasers, can be used as well.

The system and methods can use the individual absolute readings of fluorescence in the blue, green, red, or far-red range to determine the physiological condition and characteristics of plants. In addition, the system and methods can use the ratios of certain fluorescence readings in the blue, green, red, or far-red range to determine the physiological condition and characteristics of the plant. In one embodiment, a system and a method is arranged to monitor changes in Blue/Green and Red/Far Red fluorescence emitted by a plant. The system illuminates the target plant at periodic intervals, for example every 15 minutes with light of 365 nm wavelength. The system detects and records Blue/Green fluorescence and/or Red/Far-Red chlorophyll fluorescence emitted by the subject plant.

The system can review the change of each of the individual fluorescence wavelengths, and deduce from the data, changes in the physiological activity and health of the plant. However, the analysis of the ratios of the various fluorescent responses proves to be a much better indicator of plant stress rather than the absolute values. Such comparison normalizes out unanticipated conditions that cause a proportional decrease/increase in fluorescence (such as ambient illumination level change) and general changes in fluorescence levels such as during leaf growth and maturation of the crop. In one example only Blue and Green Fluorescence are monitored. The decrease of Blue to Green fluorescent ratio can be linked to stress experienced by the plant. In another example the system monitors the Blue and or Green fluorescence levels and the level of Red and/or Far-Red chlorophyll fluorescence emitted by the target plant and compares the various fluorescent ratios. If a decrease in the Blue to Red or Blue to Far Red ratio or the Green to Red or Green to Far Red ratio is detected, the system can determine that the subject plant is undergoing stress. In one example, such stresses can be caused by a drought condition. The system in the examples above may respond by alerting the operator or by initiating the supply of water to the target plant in response to the detection of stress. In another example the system may be able to identify the specific stress by analysis of the fluorescence information and alert the operator or adjust the environmental parameter accordingly (water, heat, air, nutrients, light). In yet another example the system may detect stress by analysis of the fluorescent data and identify the source by reviewing the environmental parameters of the CEA system.

The devices, systems, arrangements, and methods disclosed in this document are described in detail by way of examples. It will be appreciated that modifications to disclosed and described examples, arrangements, configurations, components, elements, apparatus, methods, materials, etc. can be made and may be desired for a specific application. In this disclosure, any identification of specific techniques, arrangements, method, etc. are either related to a specific example presented or are merely a general description of such a technique, arrangement, method, etc. Identifications of specific details or examples are not intended to be and should not be construed as mandatory or limiting unless specifically designated as such. Selected examples methods and systems for determining the health of a plant by the detection of fluorescence emitted by the plant are hereinafter disclosed and described in detail.

The systems and methods for determining the health of a plant using fluorescence emissions from the plant will be generally described in this Detailed Description, followed by specific arrangements of the systems and methods. As noted, when a plant is excited by a light source (such as an excitation light), some of the light is absorbed by the plant and subsequently emitted by the plant as fluorescence. These fluorescence emissions can be collected by an appropriate detector. The amount of fluorescence emitted over the spectral range is indicative of the physiological state of the plant, circadian rhythms, and can be indicative of the level of phenolic compounds and plant pigments. The applied excitation light is generally a shorter wavelength such as 365 nm, 405 nm, 420 nm, or 450 nm. Generally, the wavelength of the excitation light will have a shorter wavelength than the lowest bandwidth of the spectra of light the system is arranged to detect.

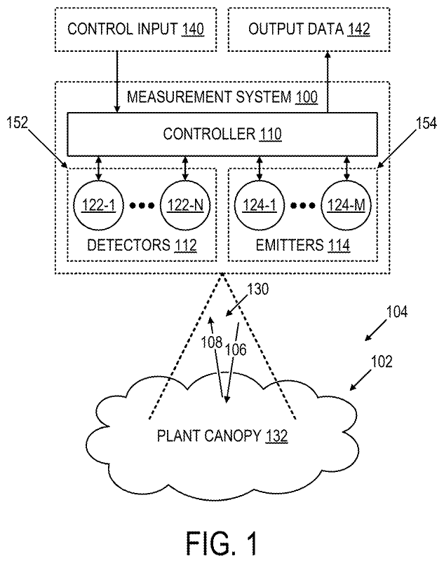

FIG. 1 is a schematic diagram depicting an example measurement system 100 for measuring fluorescence response of a subject plant 102 within an environment 104. While a subject plant is described herein for purposes of illustration, the subject plant can refer to a plurality of plants with respect to which fluorescence response is measured.

Measurement system 100 includes a controller 110, a set of one or more detectors 112 each operatively coupled with the controller via one or more communication links, and a set of one or more emitters 114 each operatively coupled with the controller via one or more communication links. Controller 110 can control and otherwise communicate with each detector of the set of detectors 112 and each emitter of the set of emitters via these communication links. Communication links between or among controller 110, the set of detectors 112, and the set of emitters 114 can include physical electrical pathways or wireless communication links.

Example detectors of the set of detectors 112 are depicted schematically in FIG. 1, including detector 122-1 through detector 122-N, where “N” represents any suitable quantity of additional detectors. For example, “N” can represent zero, one, two, three or more additional detectors. Each of detectors 122-1 through 122-N can be selectively operated by controller 110 to measure light intensity observed by that detector within an observation wavelength range. For example, each of detectors 122-1 through 122-N is configured to generate an output signal that is responsive to and based on light intensity observed by that detector within its observation wavelength range. In at least some examples, each detector of the set of detectors 112 can take the form of a photodiode. However, other suitable light detection devices or technologies can be used.

Example emitters of the set of emitters 114 are depicted schematically in FIG. 1 as emitter 124-1 through emitter 124-M, where “M” represents any suitable quantity of emitters. For example, “M” can represent zero, one, two, three or more additional emitters. Each of emitters 124-1 through 124-M can be selectively operated by controller 110 to emit light within an emission wavelength range. For example, each of emitters 124-1 through 124-N is configured to emit light of a target light intensity within an emission wavelength range that is responsive to and based on a control signal received from controller 110. In at least some examples, each emitter of the set of emitters 114 can take the form of a light emitting diode (LED). However, other suitable light emitting devices or technologies can be used.

While detectors and emitters of measurement system 100 are depicted as separate elements, in at least some examples, an individual optical element can provide both a detector function and an emitter function. In such examples, the optical element can be operated as an emitter to provide an emitter function during a first period of time, and can be operated as a detector to provide a detector during a second period of time. Where the present disclosure refers to an emitter or a detector, an optical element that is operable as both an emitter and a detector can be used. For example, where measurement system 100 is referred to as having an emitter and a detector, the measurement system 100 can include a single optical element that provides both an emitter function and a detector function at different times.

Light emitted by the set of emitters 114 can be referred to as excitation light, which can be provided to produce a fluorescence response from the subject plant 102. FIG. 1 schematically depicts an example of excitation light 106 emitted by some or all of the emitters of the set of emitters 114, and a fluorescence response 108 from subject plant 102 that takes the form of light that can be detected by some or all of the detectors of the set of detectors 112.

In at least some examples, different detectors of the set of detectors 112 can be used by controller 110 to measure light intensity within different observation wavelength ranges. For example, a first detector of the set of detectors 112 can be selectively operated by controller 110 to measure light intensity within a first observation wavelength range, and a second detector of the set of detectors 112 can be selectively operated by controller 110 to measure light intensity within a second observation wavelength range that differs from the first observation wavelength range.

Alternatively or additionally, in at least some examples, an individual detector of the set of detectors 112 can be configured to detect light intensity over a broad spectrum, and the detector can be selectively operated by controller 110 to measure light intensity within two or more different observation wavelength ranges at different times. In this scenario, the detector can be configured with a plurality of filters that can be selected by controller 110 to implement detection of light intensity within a particular wavelength range that is a subset of the broad spectrum capability of the detector. For example, detector 122-1 can be selectively operated by controller 110 to measure light intensity within a first observation wavelength range during a first observation period by selecting a first filter, and to measure light intensity within a second observation wavelength range that differs from the first observation wavelength range during a second observation period by selecting a second filter.

Alternatively or additionally, in at least some examples, a plurality of detectors of the set of detectors 112 can be selectively operated by controller 110 to concurrently measure light intensity within the same observation wavelength range during the same observation period. For example, two or more detectors of the set of detectors 112 can be selectively operated by controller 110 to concurrently measure light intensity within the same observation wavelength range during the same observation period from different locations in which each detector has a different field of view of environment 104. In this example, the different field of views of the detectors can be overlapping and contain at least a portion of subject plant 102. This approach can be used to provide stereo or multi-channel measurements of light intensity within an observation wavelength range.

In still further examples, a plurality of detectors of the set of detectors 112 can be selectively operated by controller 110 to concurrently measure light intensity within different observation wavelength ranges during the same observation period. For example, a first group of one or more detectors can be operated to measure light intensity within a red and/or near-red light wavelength range, and a second group of one or more detectors can be operated concurrently with the first group to measure light intensity within a green and/or blue light wavelength range.

In at least some examples, different emitters of the set of emitters 114 can be used by controller 110 to emit light within different emission wavelength ranges. For example, a first emitter of the set of emitters 114 can be selectively operated by controller 110 to emit light within a first emission wavelength range, and a second emitter of the set of emitters 114 can be selectively operated by controller 110 to emit light within a second emission wavelength range that differs from the first emission wavelength range.

Alternatively or additionally, in at least some examples, an individual emitter of the set of emitters 114 can be configured to emit light over a broad spectrum, and the emitter can be selectively operated by controller 110 to emit light within two or more different emission wavelength ranges at different times. In this scenario, the emitter can be configured with a plurality of filters that can be selected by controller 110 to implement emission of light within a particular wavelength range that is a subset of the broad spectrum capability of the emitter. For example, emitter 124-1 can be selectively operated by controller 110 to emit light within a first observation wavelength range during a first emission period by selecting a first filter, and to emit light within a second emission wavelength range that differs from the first emission wavelength range during a second emission period by selecting a second filter.

Alternatively or additionally, in at least some examples, a plurality of emitters of the set of emitters 114 can be selectively operated by controller 110 to concurrently emit light within the same emission wavelength range during the same emission period. For example, two or more emitters of the set of emitters 114 can be selectively operated by controller 110 to concurrently emit light within the same emission wavelength range during the same emission period from different locations in which each emitter provides a different emission field within environment 104. As an example, the set of emitters 114 can include four emitters in which each emitter takes the form of an LED that outputs light with a 365 nm wavelength. However, it will be understood that other suitable quantities of emitters and wavelengths or wavelength ranges can be used. In this example, the different emission fields of the emitters can overlap with each other and can contain at least a portion of subject plant 102. This approach can be used to provide stereo or multi-channel emissions of light within an emission wavelength range.

Within FIG. 1, for purposes of illustration, a generalized field 130 of environment 104 is depicted for measurement system 100 that represents a generalized field of view of the set of detectors 112 and a generalized emission field of the set of emitters 114. It will be understood that individual detectors of the set of detectors 112 can have a detector-specific field of view that differs from generalized field 130. It will also be understood that individual emitters of the set of emitters 114 can have an emitter-specific emission field that differs from generalized field 130.

In the example of FIG. 1, generalized field 130 includes plant canopy 132 of subject plant 102 within environment 104. For example, the detector-specific field of view of each detector of the set of detectors 112 and the emitter-specific emission field of each emitter of the set of emitters 114 can include at least a portion of plant canopy 132. In this example, the detector-specific field of view of each detector of the set of detectors 112 and the emitter-specific emission field of each emitter of the set of emitters 114 can overlap with each other.

In an example implementation of measurement system 100, the set of detectors 112 and the set of emitters 114 are located above plant canopy 130. However, it will be understood that the set of detectors 112 and the set of emitters 114 can be arranged at other suitable locations in relation to plant canopy 130 of subject plant 102. Furthermore, it will be understood that subject plant 102 can refer to a plurality of plants that collectively provide or otherwise define plant canopy 130.

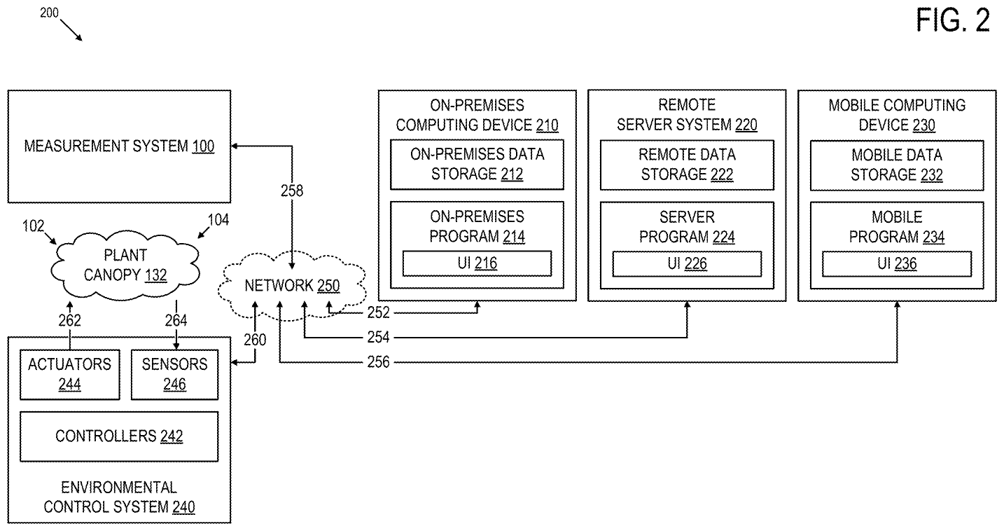

In at least some examples, as described in further detail herein with reference to FIG. 2, controller 110 can receive a control input 140 from one or more other devices, and can provide output data 142 to one or more other devices. For example, controller 110 can be configured to control the set of detectors 112 and the set of emitters 114 based on and responsive to control input 140. In at least some examples, output data 142 can include measurements of light intensity obtained by controller 110 via the set of detectors 112. Additionally or alternatively, output data 142 can include processed data that is based on and responsive to measurements of light intensity obtained by controller 110 via the set of detectors 112. In this example, controller 110 can be configured to perform processing of such measurements to obtain the processed data.

In at least some examples, the various components of measurement system 100, including controller 110, the set of detectors 112, and the set of emitters 114 can be housed within the same enclosure. In other examples, some or all of the set of detectors 112 and/or some of all of the set of emitters 114 can be housed in separate enclosures from controller 110. For example, the set of detectors 112 and the set of emitters 114 can be housed in a first enclosure and controller 110 can be housed in a second enclosure. As another example, controller 110 and the set of detectors 112 can be housed in a first enclosure and the set of emitters 114 can be housed in a second enclosure.

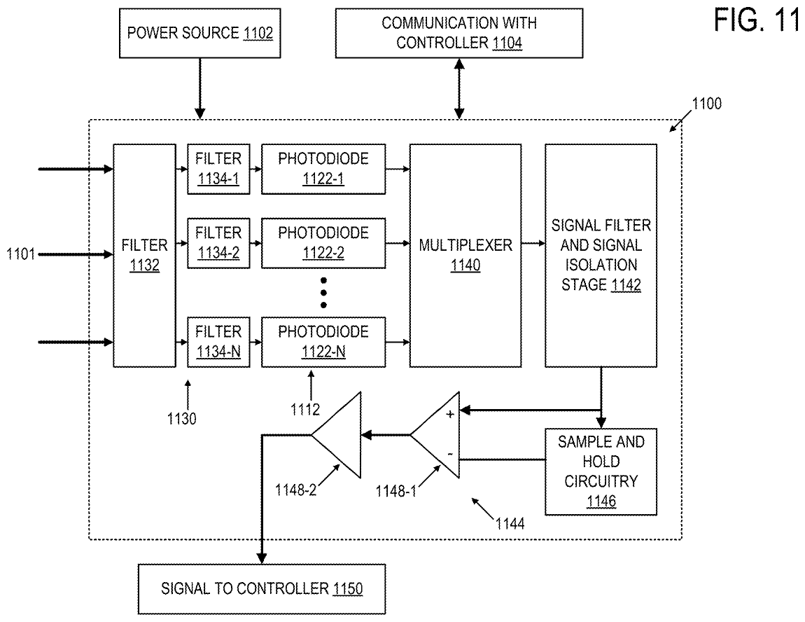

The set of detectors 112 can be combined with additional components to form a detection module 152, represented schematically in FIG. 1. Additional components of detection module 152 can include electronic components, filters, lenses, and other suitable components. An example of detection module 152 is described in further detail with reference to FIG. 11.

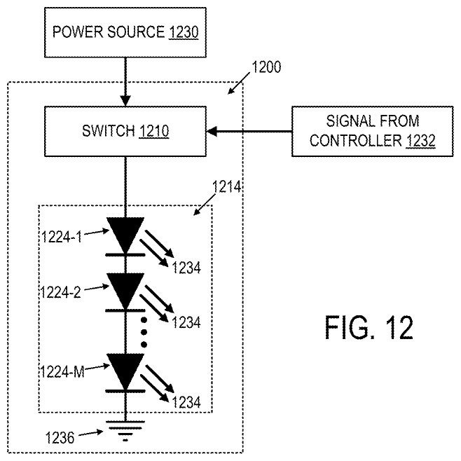

The set of emitters 114 can be combined with additional components to form an excitation module 154, represented schematically in FIG. 1. Additional components of excitation module 154 can include electronic components, filters, lenses, and other suitable components. An example of excitation module 154 is described in further detail with reference to FIG. 12.

FIG. 2 is a schematic diagram depicting an example computing system 200 that includes measurement system 100 of FIG. 1. Computing system 200 can further include an on-premises computing device 210, a remote server system 220, a mobile computing device 230, and an environmental control system 240 that can communicate with each other via a communications network 250. Communications network 250 can include one or more wide area networks (e.g., the Internet or a portion thereof), local area networks, and/or personal area networks that support communications over wired and/or wireless communications links using any suitable communications protocol or combination of protocols.

On-premises computing device 210, when included as part of computing system 200, can include on-premises data storage 212 and an on-premises program 214 (e.g., an application program) that includes a user interface (UI) 216. On-premises computing device 210 can be installed or otherwise located at the same facility where measurement system 100, environment 104, and subject plant 102 are located. In this example, on-premises computing device 210 can communicate with measurement system 100 via a personal area network and/or local area network of communications network 250, as depicted by communication links 252 and 258 in FIG. 1. Furthermore, in at least some examples, communications between measurement system 100 and on-premises computing device 210 can include control input 140 and/or output data 142, previously described with reference to FIG. 1. For example, control input 140 can be provided by on-premises program 214, and output data 142 can be stored at on-premises data storage 212 and can be processed by on-premises program 214.

Remote server system 220, when included as part of computing system 200, can include remote data storage 222 and a server program 224 (e.g., an application program) that includes a user interface 226. Within the context of a server, user interface 226 and server program 224 can be accessed via a web browser from another computing device over communications network 250, as an example. Remote server system 220 includes one or more server computing devices that are remotely located from the facility where measurement system 100, environment 104, and subject plant 102 are located. In this example, remote server system 220 can communicate with measurement system 100 via at least a wide area network of communications network 250, as depicted by communication links 254 and 258. Furthermore, in at least some examples, communications between measurement system 100 and remote server system 220 can include control input 140 and/or output data 142, previously described with reference to FIG. 1. For example, control input 140 can be provided by server program 224, and output data 142 can be stored at remote data storage 222 and can be processed by server program 224.

Mobile computing device 230, when included as part of computing system 200, can include mobile data storage 232 and a mobile program 234 (e.g., an application program) that includes a user interface 236. Mobile computing device 230 can communicate with measurement system 100 via one or more of a wide area network, local area network, and/or personal area network of communications network 250, as depicted by communication links 256 and 258. In at least some examples, communications between measurement system 100 and mobile computing device 230 can include control input 140 and/or output data 142, previously described with reference to FIG. 1. For example, control input 140 can be provided by mobile program 234, and output data 142 can be stored at mobile data storage 232 and can be processed by mobile program 234.

Environmental control system can be configured to control one or more operating conditions of environment 104 containing subject plant 102. Environmental control system 240 can include one or more controller 242, one or more physical actuators 244, and one or more sensors 246. Physical actuators 244 can be selectively operated by the one or more controllers 242 to control one or more operating conditions of environment 104, for example, by varying an aspect of one or more environmental inputs 262 to environment 104, as will be described in further detail herein. The one or more controllers 242 can measure one or more conditions of environment 104 via the one or more sensors 246. Environmental control system 240 can communicate with measurement system 100 via one or more of a local area network and/or a personal area network of communications network 250, as depicted by communications links 258 and 260. In at least some examples, communications between measurement system 100 and environmental control system 240 can include control input 140 and/or output data 142, previously described with reference to FIG. 1. For example, control input 140 can be provided by the one or more controllers 242, and output data 142 can be stored at and/or processed by the one or more controllers 242 environmental control system 240.

Computing system 200 can support a variety of additional or alternative modes of communications between or among on-premises computing device 210, remote server system 220, mobile computing device 230, and environmental control system 240 that traverse one or more intermediate devices.

As a first example, communications between measurement system 100 and mobile computing device 130 (e.g., communications that include control input 140 and output data 142) can traverse one or more intermediate devices, including on-premises computing device 110 and/or server system 120. For example, mobile computing device 130 can receive output data 142 or processed forms thereof from remote data storage 222 of server system 220 or from on-premises data storage 112 of on-premises computing device 110 via communications network 250. As another example, mobile computing device 130 can send or initiate the sending of control input 140 to measurement system 100 by communicating with server program 224 of server system 220 or with on-premises program 214 of on-premises computing device 210 via communications network 250.

As a second example, communications between environmental control system 240 and mobile computing device 130 can traverse one or more intermediate devices, including on-premises computing device 110 and/or server system 120. For example, mobile computing device 130 can receive sensor data or processed forms thereof originating from sensors 246 of environmental control system 240 from remote data storage 222 of server system 220 or from on-premises data storage 112 of on-premises computing device 110 via communications network 250. As another example, mobile computing device 130 can send or initiate the sending of a control input to actuators 244 of environmental control system 240 by communicating with server program 224 of server system 220 or with on-premises program 214 of on-premises computing device 210 via communications network 250.

As a third example, communications between environmental control system 240 and measurement system 100 can traverse one or more intermediate devices via communications network 250, including on-premises computing device 110, server system 120, and/or mobile computing device 130. As an example, output data 142 from measurement system 100 can be received by server system 120 where the output data is processed to generate commands that are sent to environmental control system 240. As another example, sensor data obtained from sensors 246 of environmental control system 240 can be received by server system 120 where the sensor data is processed to generate commands that are sent to measurement system 100 as control input 140.

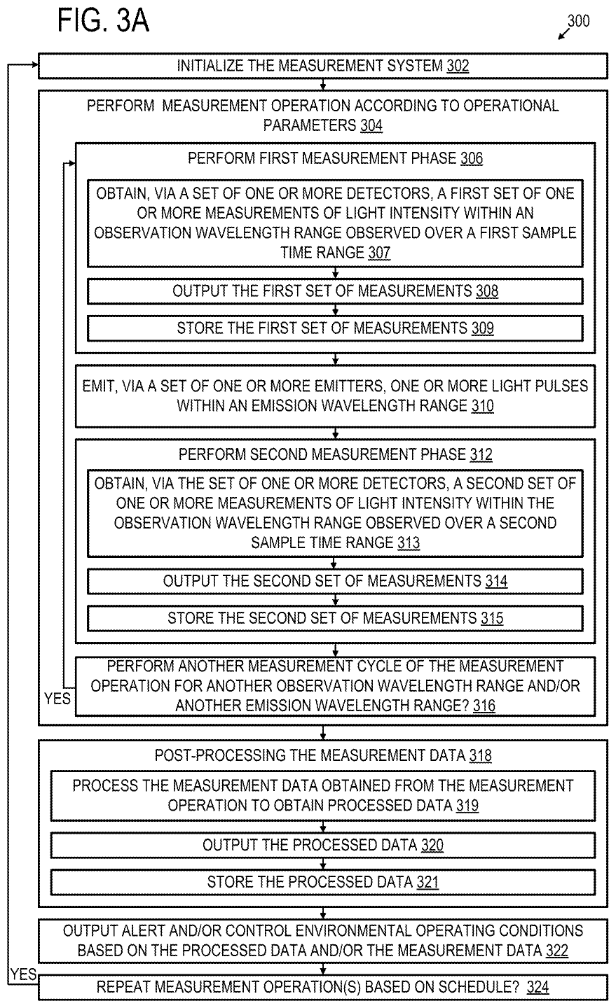

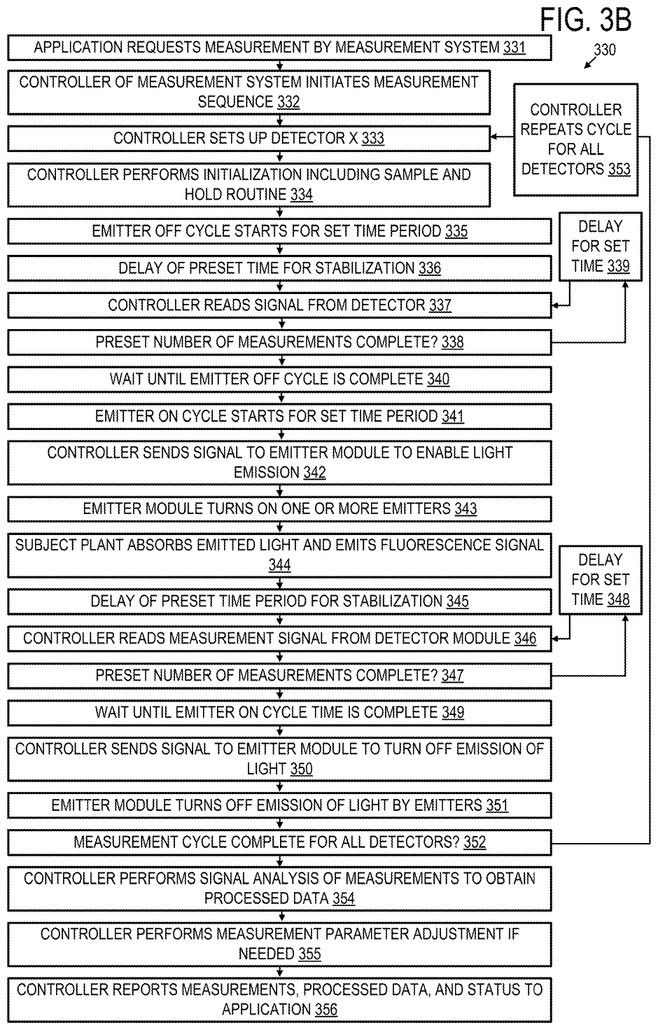

As described in further detail with reference to FIG. 3, alerts can be output by one or more of controller 110 of measurement system 100, on-premises computing device 210, remote server system 220, mobile computing device 230, and/or controllers 242 of environmental control system 240 responsive to measurement data or processed data obtained from the measurement data satisfying a criteria that is indicative of a particular plant status. For example, alerts can be sent from a device or system that outputs the alert to another device or system of computing system 200 to notify staff or technicians that a particular subject plant or set of plants is experiencing a particular plant status, such as a status indicative of intervention due to deteriorating health, increased stress, or physiological condition.

While computing system 200 depicts a single instance of measurement system 100, in at least some examples, computing system 200 can include many instances of measurement system 100 that are distributed within a facility or plant growing environment to measure and monitor plant status of respective subject plants.

FIG. 3 is a flow diagram depicting an example method 300 of monitoring plant status, which can include plant health, plant stress, plant physiological condition, as examples. Method 300 can be performed by a computing system of one or more computing devices. In at least some examples, method 300 can be performed by computing system 200 of FIG. 2, including at least measurement system 100.

At 302, the method includes initializing the measurement system, such as measurement system 100 of FIG. 1, as an example. Initialing the measurement system at 302 can include, as examples, loading operational parameters and/or a measurement schedule onto controller 110 of measurement system 100, and operating the emitters and detectors, and associated circuitry of the measurement system according to an initialization procedure to ensure satisfactory operation. As an example, a gain of one or more signal amplifiers of the detector module may be set to a target value (e.g., 80% of maximum). As another example, sample and hold circuitry of the detector module can be charged. As yet another example, a multiplexer of the detector module can be set to multiplex signals for some or all of the detectors.

At 304, the method includes performing a measurement operation according to the operational parameters. As described in further detail herein, the operational parameters can define various aspects of the measurement operation, including a quantity of measurements obtain during the measurement operation, observation wavelength ranges at which detectors are to measure light intensity, a frequency at which measurements are obtained during the measurement operation, a duration of time of a sample period of each measurements, a duration of time between measurements, an emission wavelength range at which emitters are to emit excitation light, a quantity of light pulses of the excitation light, a duration of the light pulses, a duration of time between the light pulses, a delay between measurement operations, and a relative timing of measurements obtained by the detectors relative to the light pulses emitted by the emitters.

As part of the measurement operation performed at 304, the method includes performing a first measurement phase at 306. As part of the first measurement phase performed at 306, the method includes obtaining, via a set of one or more detectors (e.g., 112 of FIG. 1), a first set of one or more measurements of light intensity within an observation wavelength range at 307. In at least some examples, each measurement of the first set of measurements is based on light intensity observed by the set of one or more detectors within the observation wavelength range over a first sample time range. The first sample time range can correspond to a duration of time over which the light intensity is sampled and/or held. For example, sample and hold circuitry of the measurement system can be implemented to obtain each measurement as an integral of light intensity within the observation wavelength range observed over the first sample time range. Where the first set of measurements includes two or more measurements, the first set of measurements can form a sequence of measurements that are each captured over a respective instance of the first sample time range that is spaced apart in time from neighboring measurements of the sequence by a duration of time. The duration of time can be the same between each neighboring pair of measurements.

As part of the first measurement phase performed at 306, the method can further include outputting, at 308, the first set of measurements obtained at 307 and/or storing the first set of measurements at 309 in data storage.

As part of the measurement operation performed at 304, the method includes emitting, via a set of one or more emitters (e.g., 114 of FIG. 1), one or more light pulses within an emission wavelength range. Where two or more light pulses are emitted as part of operation 310, each light pulse can be spaced apart from neighboring light pulses by a period of time during which the emitters do not emit light or do not emit light within the emission wavelength range and the observation wavelength range used at 307. Where two or more light pulses are emitted as part of operation 310, the light pulses can be emitted as a sequence of light pulses in which each light pulse of the sequence is spaced apart from neighboring light pulses by the same period of time.

As part of the measurement operation performed at 304, the method includes performing a second measurement phase at 312. As part of the second measurement phase performed at 312, the method, at 313, includes obtaining, via the set of one or more detectors, a second set of one or more measurements of light intensity within the observation wavelength range for which the first set of measurements were obtained at 307. In at least some examples, each measurement of the second set of measurements is based on light intensity observed by the set of one or more detectors within the observation wavelength range over a second sample time range. The second sample time range can be the same as the first sample time range used to obtain the first set of measurements at 307. The second sample time range can correspond to a duration of time over which the light intensity is sampled and/or held. For example, sample and hold circuitry of the measurement system can be implemented to obtain each measurement as an integral of light intensity within the observation wavelength range observed over the second sample time range. Where the second set of measurements includes two or more measurements, the second set of measurements can form a sequence of measurements that are each captured over a respective instance of the second sample time range that is spaced apart in time from neighboring measurements of the sequence by a duration of time. The duration of time can be the same between each neighboring pair of measurements. Furthermore, the duration of time can be the same as the duration of time between each neighboring pair of the first set of measurements captured at 307.

As part of the second measurement phase performed at 312, the method can further include outputting, at 314, the second set of measurements obtained at 313 and/or storing the second set of measurements at 315 in data storage.

The first measurement phase performed at 306, the emission of one or more light pulses performed at 310, and the second measurement phase performed at operation 312 collectively form a measurement cycle of the measurement operation performed at 304. In at least some examples, one or more additional measurement cycles can be performed as part of the measurement operation. Each additional measurement cycle can be used to obtain measurements of light intensity within a different observation wavelength range. Additionally or alternatively, each additional measurement cycle can be used performed a different emission wavelength range.

At 316, the method includes determining whether another measurement cycle of the measurement operation is to be performed for another observation wavelength range and/or another emission wavelength range. If another measurement cycle is to be performed, the method can return to operation 306 where the first measurement phase is performed, emission of light pulses is performed at 310, and the second measurement phase is performed at 312 for the additional measurement cycle, for example, using a different observation wavelength range and/or a different emission wavelength range in relation to a prior measurement cycle of the measurement operation. Thus, for each measurement cycle of the measurement operation, a first set of measurements and a second set of measurements are obtained, output and/or stored. If another measurement cycle is not to be performed at 316, the method can proceed to 318.

At 318, the method includes post processing the measurement data obtained from the measurement operation performed at 304 over one or more measurement cycles. As part of post-processing performed at 318, the method includes processing the measurement data obtained from the measurement operation to obtained processed data. The processed data can be output at 320 and/or stored in data storage at 321. As an example of processing performed at 319, multiple measurements obtained for a given measurement phase can be averaged. As another example, measurements or averages of measurements obtained for a first observation wavelength range can be subtracted from measurements or averages of measurements obtained for a second observation wavelength range to remove background fluorescence from a resulting measurement of fluorescence that is indicative of the plant status of the subject plant or set of plants. The plant status can refer to one or more of a health, stress, or physiological condition of the subject plant or plants, as examples.

Furthermore, in at least some examples, the resulting measurement of fluorescence obtained from measurement operations periodically conducted over a period of time can be compared to each other and to various criteria to determine whether an alert is to be output and/or environmental operating conditions of the environment are to be varied or otherwise controlled. As a first example, an alert is output responsive to any of the resulting measurement of fluorescence exceeding a threshold value. A second example, a control signal to one or more physical actuators to control an operating condition of the environment containing the one or more plants is output responsive to any of the resulting measurement of fluorescence exceeding a threshold value. As a third example, processing of the measurement data performed at 319 can include identifying a rate of change of the resulting measurement of fluorescence over the period of time that multiple measurement operations are conducted, the rate of change can be compared to a threshold rate of change, and an alert is output responsive to the rate of change exceeding the threshold rate of change. As a fourth example, processing of the measurement data performed at 319 can include identifying a rate of change of the resulting measurement of fluorescence over the period of time that multiple measurement operations are conducted, the rate of change can be compared to a threshold rate of change, and a control signal to one or more physical actuators is output to control an operating condition of the environment containing the one or more plants responsive to the rate of change exceeding the threshold rate of change. The various thresholds of the preceding examples can be stored within data storage as rule sets, as an example.

At 322, the method includes outputting an alert (e.g., to one or more other devices, such as computing devices hosting an application program) and/or controlling one or more environmental operating conditions based on the processed data and/or the measurement data. Example operating conditions that can be controlled or otherwise varied include one or more of: a quantity of water supplied to the environment, a frequency at which water is supplied to the environment, an ambient temperature of the environment, an intensity of light supplied to the environment, a duration of light supplied to the environment, a spectral composition of the light supplied to the environment, an ambient humidity of the air of the environment, a quantity of a nutrient supplied to the environment, a type of nutrient supplied to the environment, a frequency at which a nutrient is supplied to the environment.

At 324, one or more measurement operations may be repeated based on a schedule, in which case the method can proceed to 302 where initialization is performed.

FIG. 3B is a flow diagram depicting an example method 330. Method 330 includes an example implementation of method 300 of FIG. 3A. At 331, the method includes an application program (e.g., 214, 224, 234) executed by a computing device (e.g., 210, 220, 230) requesting that a measurement operation be performed by the measurement system (e.g., 100). At 332, the method includes the controller (e.g., 110) of the measurement system (e.g., 100) initiating a measurement sequence. At 333, the method includes the controller setting up a detector, identified by the variable “X”, where X can refer to any of the detectors of the set of detectors (e.g., 112). At 334, the method includes the controller performing initialization that includes performing a sample and hold routine. At 335, the method includes starting the emitter off cycle for a set time period. At 336, the method includes delaying measurement of light intensity by the detector by a preset time to allow for stabilization of the detection circuitry, including the detector and the sample and hold circuit. At 337, the method includes the controller reading the signal from the detector. At 338, the method includes the controller determining whether the preset number of measurements have been completed. If the preset number of measurements has not been completed, the method can including delaying a subsequent measurement for a set time at 339 and can return to 337. If the preset number of measurements has been completed, the method proceeds to 340. At 340, the method includes the controller waiting until the emitter off cycle is complete. At 341, the method includes starting the emitter on cycle for the set time period. At 342, the method includes the controller sending a signal to the emitter module (e.g., 154) to enable emission of light by one or more emitters. At 343, the method includes the emitter module turning on the one or more emitters for a preset period of time to emit light before discontinuing emission of light. At 344, the method includes the subject plant absorbing emitted light and the subject plant emitting a fluorescence signal. At 345, the method includes delaying measurement of light by the detector for a preset period of time for stabilization of the system. At 346, the method includes the controller reading the measurement signal from the detector module. At 347, the method includes determining whether a preset number of measurements is complete. If the preset number of measurements is not complete, the method includes delaying the subsequent measurement for a set period of time, and the method returns to 346. If the preset number of measurements is complete, the method proceeds to 349. At 349, the method includes the controller waiting until an emitter on cycle time is complete. At 350, the method includes the controller sending a signal to the emitter module to turn off the emission of light by the emitters. At 351, the method includes the emitter module turning off the emission of light by the emitters. At 352, the method includes determining whether the measurement cycle is complete for all detectors. If the measurement cycle is not complete for all detectors, the method can proceed to 353 where the controller repeats the measurement cycle for all detectors by returning to 333 for a next detector. If the measurement cycle is complete for all detectors, the method proceeds to 354. At 354, the method includes the controller performing signal analysis (i.e., processing) of the measurements to obtain processed data. At 355, the method includes the controller performing measurement parameter adjustment if needed, for example, to adjust operational parameters for subsequent measurements. At 356, the method includes the controller reporting the measurements, the processed data, and/or status to the application program.

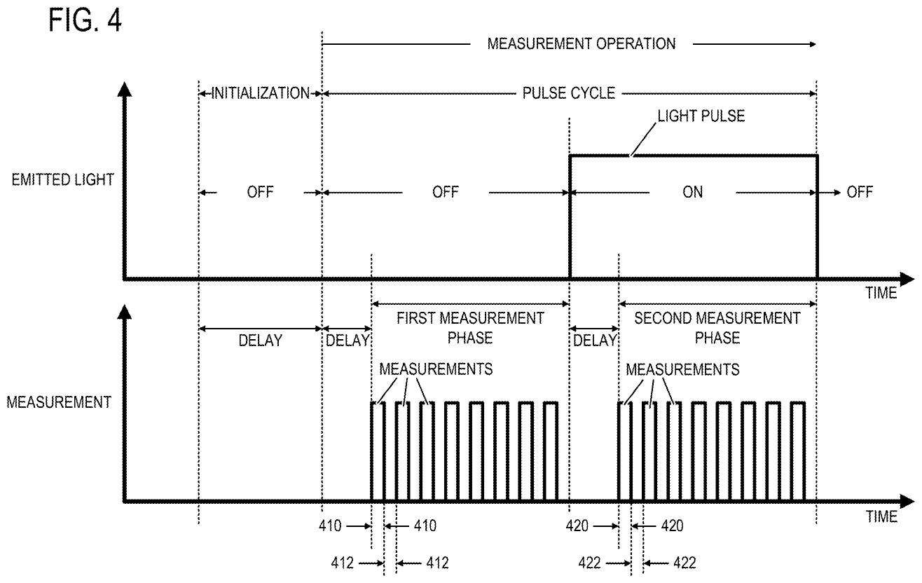

FIG. 4 depicts a timeline in which light emitted by one or more emitters of measurement system 100 of FIG. 1 is varied over time between an off state and an on state according to operation 310 of method 300 of FIG. 3. The timeline of FIG. 4 further depicts measurements of light intensity obtained by one or more detectors of measurement system 100 of FIG. 1 over time according to an implementation of method 300 of FIG. 3. In FIG. 4, initialization is performed as described with reference to operation 302 of method 300 prior to a measurement operation being performed. In this example, light is not emitted by the emitters (i.e., off) and measurements are not obtained (i.e., delay) during initialization. During initialization, a gain of the detectors can be set, as previously described with reference to method 300. Additionally, delaying the capture of measurements during initialization can be used to enable charging of the sample and hold circuitry that is used to measure light intensity over a sample period of time.

Following initialization, the measurement operation is performed, such as described with reference to operation 304 of FIG. 3. In this example, a portion of the measurement operation is depicted for a pulse cycle that includes an initial period of time where the emitters do not emit light (i.e., off) followed by a subsequent period of time where a light pulse is emitted by the emitters (i.e., on) according to operation 310 of method 300. The light emitted by the emitters as a light pulse is concluded at the end of the pulse cycle when emission of light by the emitters is discontinued (i.e., off). As described with reference to method 300, the measurement operation can include additional light pulses as part of operation 310, for example, by one or more additional pulse cycles.

While the emitters are not emitting light during an initial period of time of the pulse cycle, the first measurement phase can be performed according to operation 306 of method 300. In this example, the first measurement phase is delayed by a period of time following onset of the pulse cycle. Following this delay, the first measurement phase is performed that includes obtaining a first set of one or more measurements of light intensity. In the example of FIG. 4, the first measurement phase includes eight measurements of light intensity that forms a sequence of measurements. It will be understood that other suitable quantity of measurements can be obtained. During the first measurement phase, the sample and hold circuitry can be enabled by the controller. The measurements of light intensity obtained for the first measurement phase can be used to capture a baseline or background fluorescence observed by the detectors prior to emission of the light pulse.

Each measurement of the set of measurements obtained for the first measurement phase captures light intensity observed by the detectors over a first sample time range 410, depicted in FIG. 4 with respect to an example measurement. Each measurement of the sequence of measurements obtained during the first measurement phase is spaced apart in time from neighboring measurements of the sequence by a duration of time 412. The duration of time 412 can be the same between each neighboring pair of measurements of the sequence.

In the example of FIG. 4, the first measurement phase concludes before the pulse of light is emitted by the emitters. The second measurement phase is performed according to operation 312 of method 300 following the first measurement phase. In the example of FIG. 4, the second measurement phase is delayed by a period of time following onset of the pulse of light emitted by the emitters. Following this delay, the second measurement phase is performed that includes obtaining a second set of one or more measurements of light intensity. In the example of FIG. 4, the second measurement phase includes eight measurements of light intensity that forms a sequence of measurements. It will be understood that other suitable quantity of measurements can be obtained. During the second measurement phase, the sample and hold circuitry can be enabled by the controller. The measurements of light intensity obtained for the second measurement phase can capture a fluorescence response of the subject plant to the light pulse.

Each measurement of the set of measurements obtained for the second measurement phase captures light intensity observed by the detectors over a second sample time range 420, depicted in FIG. 4 with respect to an example measurement. The second time range 420 can be the same as the first sample time range 410 of the first set of measurements obtained during the first measurement phase. Each measurement of the sequence of measurements obtained during the second measurement phase is spaced apart in time from neighboring measurements of the sequence by a duration of time 422. The duration of time 422 can be the same between each neighboring pair of measurements of the sequence. Furthermore, the duration of time 422 can be the same as the duration of time 412 of the first set of measurements obtained during the first measurement phase. In the example of FIG. 4, the second measurement phase concludes before the light pulse is concluded. Upon conclusion of the pulse cycle, one or more additional instances of the pulse cycle can be performed as part of the measurement operation.

In at least some examples, an instance of the pulse cycle can be performed for each detector or group of detectors that is configured to measure light intensity within a different wavelength range. In such examples, where two or more wavelength ranges are to be measured using two or more instances of the pulse cycle, the initialization phase can be performed prior to each pulse cycle to set the gain of the detectors used in that pulse cycle and to enable charging of the sample and hold circuitry.

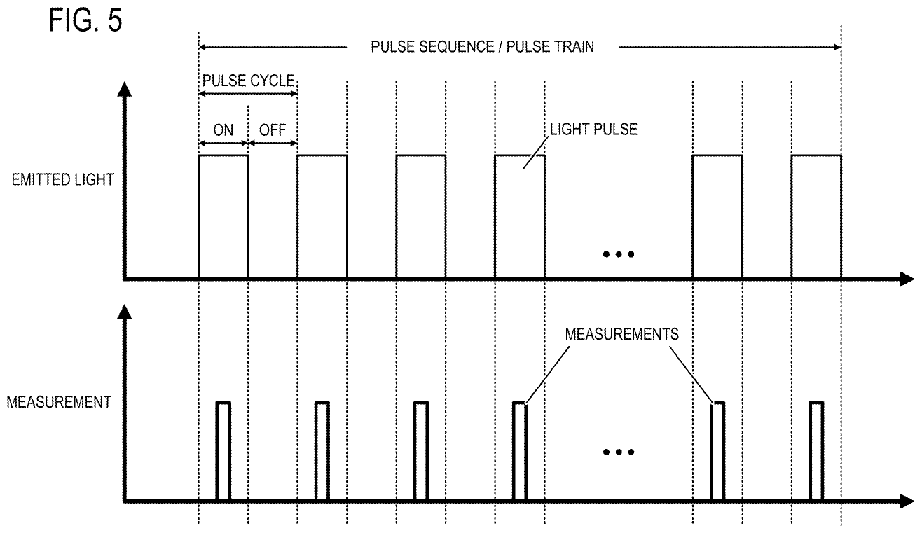

FIG. 5 depicts a timeline of another example measurement operation that can be performed by the measurement system of FIGS. 1 and 2. In FIG. 5, a sequence of light pulses, also referred to as a pulse train are output by one or more emitters of the measurement system. In this example, measurements of light intensity are performed during each light pulse.

In some embodiments (e.g., FIG. 5) each sampling of the health status includes a pulse train of excitation light. The pulse train may range from 100 microseconds to several minutes, but not be limited to this range. In one embodiment the pulse train may be 2 seconds. During the pulse train the excitation light may be turned on and off at a frequency of 10-1000 Hz, but not limited to this range. In one embodiment the frequency may be 200 Hz. In another embodiment the frequency may be 30 Hz or 60 Hz in order to cancel out 60 Hz noise. The duty cycle of the pulse may be 50%, but another value may be chosen also.

In one embodiment the frequency of the excitation light is 200 Hz (5 milliseconds duration) and the fluorescence is sampled at 400 Hz (2.5 milliseconds). The fluorescence is hence measured at least once during the on time of the excitation light and once during the off time of the excitation light. By subtracting the off signal from the on signal the background signal is removed. Repeating this measurement for a duration of 2 s provides 800 measurements that can be averaged to remove background noise further. The selection of the frequency, duty cycle and duration of the pulse is not limited to the aforementioned example. These parameters may be chosen to specifically compensate for noise present in the system and environment.

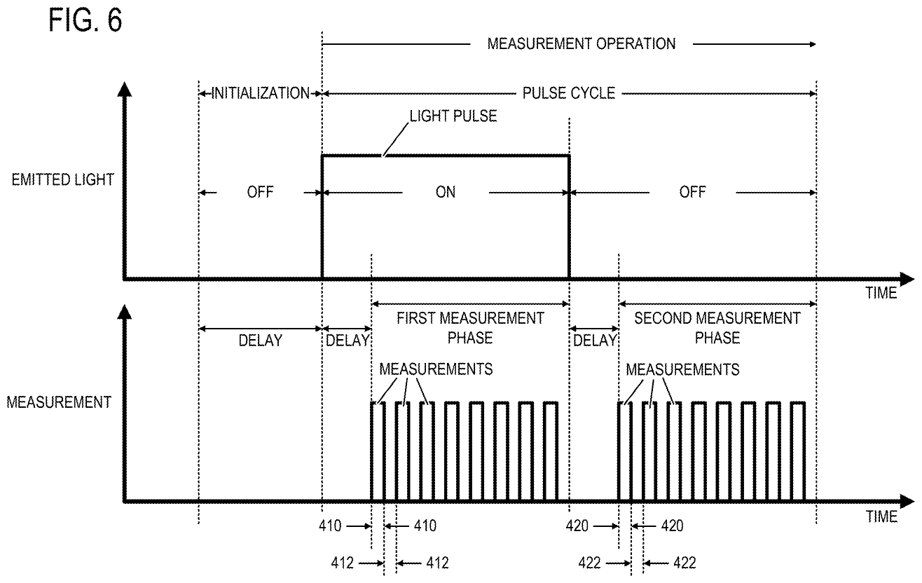

FIG. 6 depicts a timeline of another example measurement operation that can be performed by the measurement system of FIGS. 1 and 2. The timeline of FIG. 6 depicts an example in which the first measurement phase is conducted during the light pulse of each pulse cycle, and the second measurement phase is conducted during the off portion of each pulse cycle. Furthermore, in the example of FIG. 6, initialization, delays, and instances of first sample time range 410 of measurements during the first measurement phase, duration of time 412 between measurements of the first measurement phase, second sample time range 420 of measurements during the second measurement phase, duration of time 422 between measurements of the second measurement phase are depicted, such as previously described with reference to FIG. 4.

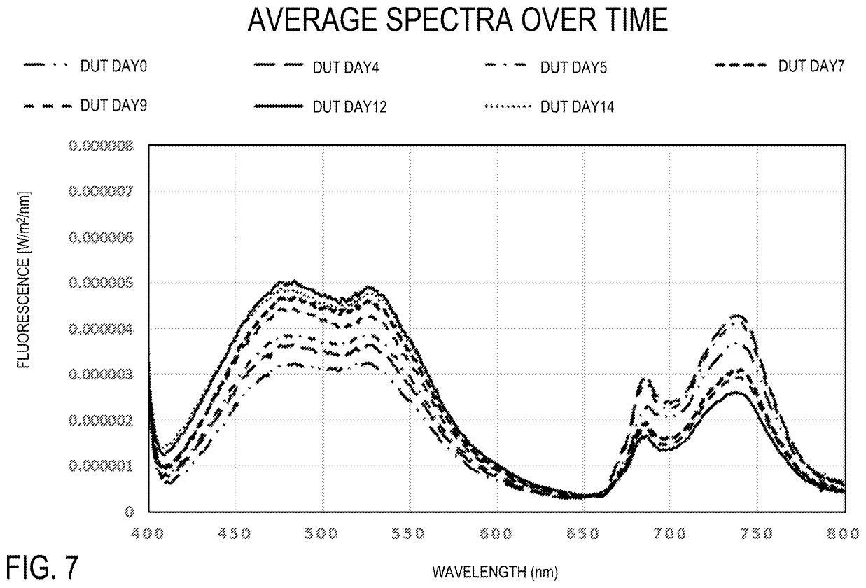

In an embodiment (e.g., FIG. 7) the frequency of the excitation light is 30 Hz at 50% duty cycle. This results in an “on time” of the excitation light of 16.7 ms, an entire 60 Hz pulse duration. The fluorescence signal is sampled in 100 microsecond intervals over the period of 16.7 ms. The signal may be stored to the cloud for post processing and/or may be averaged. This process effectively removes 60 Hz noise on the signal. The measurement is repeated for the “off time”. The “off time” signal can be subtracted from the “on time” signal to remove the background signal. This measurement may be performed for one 30 Hz cycle, several or many. Larger sampling numbers and averaging of the data may result in improved signal level and reduced noise. It is understood that frequencies, sampling intervals and sampling numbers may be adjusted as desired and required in the application.

In some embodiments a delay in the sampling of the fluorescence emission is implemented in order to compensate for stabilization of the current level and emission of the excitation source, as well as fluorescence emissions. The delay may be 100 microseconds up to hundreds of milliseconds.

The present disclosure describes various components and the operation of a system to remotely detect plant stress, health, circadian rhythms and growth dynamics via fluorescent measurements. The system comprises an Excitation module, a Detector module and Control module. The Control module writes the data to a local server or cloud storage. An application software performs the data analysis and provides the operator information on the plant health and status and may communicate with other CEA components to control the environment. The Excitation module provides the excitation light to the plants. The Excitation module may comprise one or more LEDs that emit UVA light of a wavelength of 365 nm peak wavelength. The photon flux density can range from 10 μmol/m2/s to 2000 μmol/m2/s. In one exemplary embodiment the flux density may be chosen to be below 150 μmol/m2/s, for example 75 μmol/m2/s. It is understood that the photon flux density may vary from plant to plant and application to application and that the system may be designed to have variable flux density.

It is also understood that the wavelength of the Excitation module can be any wavelength that is readily available as a product and shorter than the lowest fluorescent wavelength aimed to be detected, such as 365 nm, 405 nm, 420 nm or 450 nm. It is also understood that the excitation light can be generated by one or more lasers (VCSEL or edge emitting). A laser provides a relatively high intensity excitation light focused on a small area, which can be beneficial for the detection of fluorescence in a portion of a plant.

The Excitation module may also comprise one or more optical components. Optical elements such as hollow reflective or transparent solid lenses with suitable performance may be utilized to gather the excitation light from the source and shape the emitted beam to provide uniform illumination over a desired area. The Excitation module for example may provide significant uniform illumination of 75 μmol/m2/s at a target distance of approximately 500 mm over a circle of 150 mm diameter or 300 mm diameter. The Excitation module may also comprise an optical short pass filter to remove any light emission from the beam that is above a certain wavelength. In one embodiment the filter will have a cut off wavelength of 400 nm and an optical density of 3 or larger.

The fluorescence signal is in general a weak on the order of a 0.1 to several percent of the excitation light. Unless the sampling of the fluorescence response is performed in dark environment the system needs to also consider the influence of the room light, specifically the grow light on the detection system. The room light/grow may also cause additional undesired fluorescent signal.

In one embodiment the sampling of the fluorescence response occurs in a dark environment. In another embodiment the sampling of the fluorescence response occurs under room light/grow light and the detection and control system is designed to remove stray light and fluorescence caused by the room light/grow light.

In one embodiment the Control module may comprise an optic to limit the field of view (FOV) of the photodiodes. The field of view may match the area that is uniformly illuminated by the Excitation module. The FOV may be around 15 deg FWHM or 30 deg FWHM, for example.

In addition, the detector module may comprise a long pass window removing significantly all of the emission from the excitation source from the beam incident on the photodiodes.

In one embodiment the detection module comprises an additional “dark” photodiode, that is not exposed to any fluorescence emitted by the plant. This “dark” photodiode is used to detect background noise on the PCB that is then subsequently subtracted from the signal of the other photodiodes used to detect the fluorescence.

In one embodiment the detector module may be separate from the Control module and pass the fluorescence signal in analog or digital state to the Control module.

In a different embodiment the Detector module may be integrated with the Control module and located on the same PCB.

The Control module controls and schedules the level and timing of the excitation light as well as the sampling of the fluorescence light. The Control module may also read electronic signals provided of the fluorescence measurement by the Detector module and perform some further analysis. The Control module may also write the data gathered to a local server or a cloud location.

The Control module may contain one or more processors, microprocessors, PIC processors or the like in order to perform the above described functions as is known to a person skilled in the Art.

The Control module may use wired control protocols such as USB or UART or wireless protocols such as WiFi, Zigbee, Bluetooth, Thread or others to write the gathered information to the cloud as known to a person skilled in the Art.

In one embodiment the controller of the measurement system may comprise the “Particle Argon” kit which features the Nordic nRF52840 and Espressif ESP32 processors.

In several embodiments the health of the plant is monitored over the entire growth period. The health status of the plant may be sampled periodically in intervals of 15 minutes for example. It may of course also be of value to sample the health status more frequently, for example every minute or less frequently, for example every hour or twice or once a day. The period of the sampling will depend on the plant and the application.

It is understood that the local server or cloud storage may gather information from several, tens or a multitude of sensors at one or more growth facilities.

The system may further include an application software that provides user access to the data stored either locally or in a cloud-based application. The application software can perform additional analyses of the signal and provides the user with the physiological condition and characteristics of the target plant(s) along with historical information. The software may plot the health status or characteristics of the plant over time.

In some embodiments the software application can process, analyze, and present data gathered by a multitude of sensors installed in one or more facilities and provide the user with access to data across multiple facilities.

In some embodiments the application software may alert the operator of the detection of plant stress.

In some other embodiments the application software is able to identify the type of stress. This identification of the cause of plant stress may be possible by communication of the software with other components of the CEA (nutrient, water, air, temperature, relative humidity, light). The identification may also be possible by analyzing the fluorescence information.

In some further embodiments the application software may initiate the corrective measure itself. For example, if drought is detected the application software may initiate irrigation of the target plants.

In some embodiments, the measurement system is placed vertically above the target plants at a distance of about 300-500 mm as is typical in CEA applications. The Excitation module, Detection module and Control module may be entirely located in one housing. In another embodiment the Excitation module is separate from the Detection and Control module and the Detection and Control module and excitation module may be arranged at an angle to the plant.

The following example is a description of a specific system and method for detecting a drought condition in a plant. The objective of the example was to show that drought stress can indeed be measured with the proposed system. The system was tested by comparing several plants that were subjected to a drought condition with several control plants that were provided a sufficient water supply. The plants were placed in a controlled environment and exposed to 19 hours a day of 400 μmol/m2/s of white light for fourteen days. The soil moisture of the plants was measured daily and kept constant for the control plants while the stressed plants were not watered. The system includes an excitation source that provides 75 μmol/m2/s UVA illumination (UV LEDs with peak wavelength at 365 nm). The excitation source includes also optical short pass filters to remove illumination above 400 nm. A spectrometer was used to detect fluorescence responses from the plants. Optical long pass filters are placed in front of the spectrometer probe to effectively remove signals below 400 nm specifically the excitation source signal. The fluorescence measurement was performed once a day on each plant. For the measurement, the plant was transferred into a controlled dark environment test station. The plant was exposed to the UV excitation source for about 5 minutes during which time the fluorescence measurements were performed.

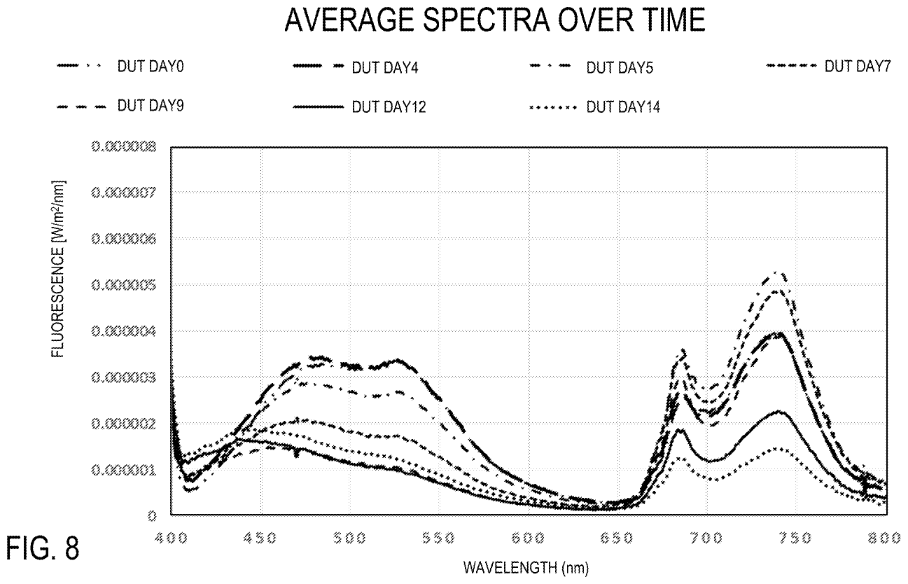

FIG. 7 is a graph showing the average fluorescence spectra for the controlled plants at 0, 4, 5, 7, 9, 12, and 14 days. FIG. 8 is a graph showing the average fluorescence spectra for the stressed plants at 0, 4, 5, 7, 9, 12, and 14 days. As illustrated by FIGS. 7 and 8, the spectral shape of the control plants remained significantly constant over the fourteen-day period, while the spectral shape of the stressed plants changed significantly over that period.

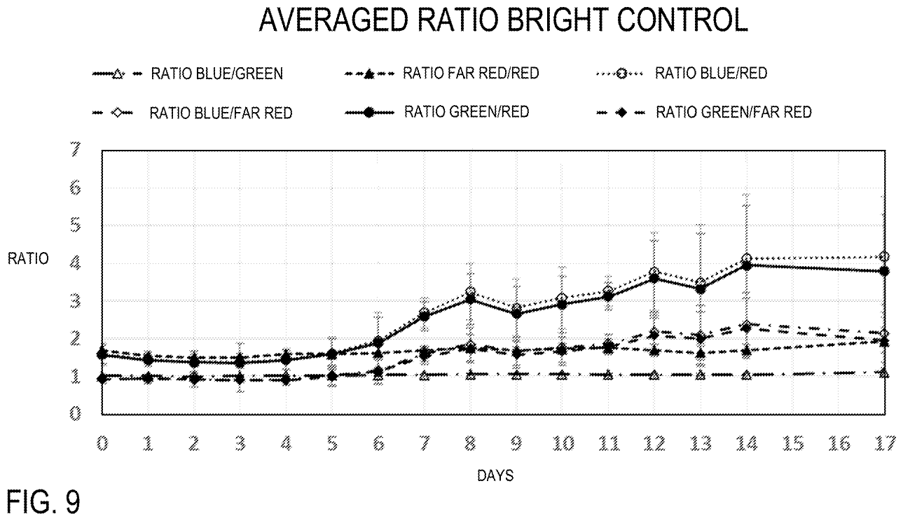

FIG. 9 illustrates average ratios of different bandwidths of detected fluorescence over time for the control plants. As is illustrated, the Blue to Green ratio is very stable and the Far-Red to Red ratio is fairly stable. The Blue to Red, the Blue to Far-Red, the Green to Red, and Green to Far Red ratios increase as blue and green fluorescence increases and red and far-red fluorescence decreases over time.

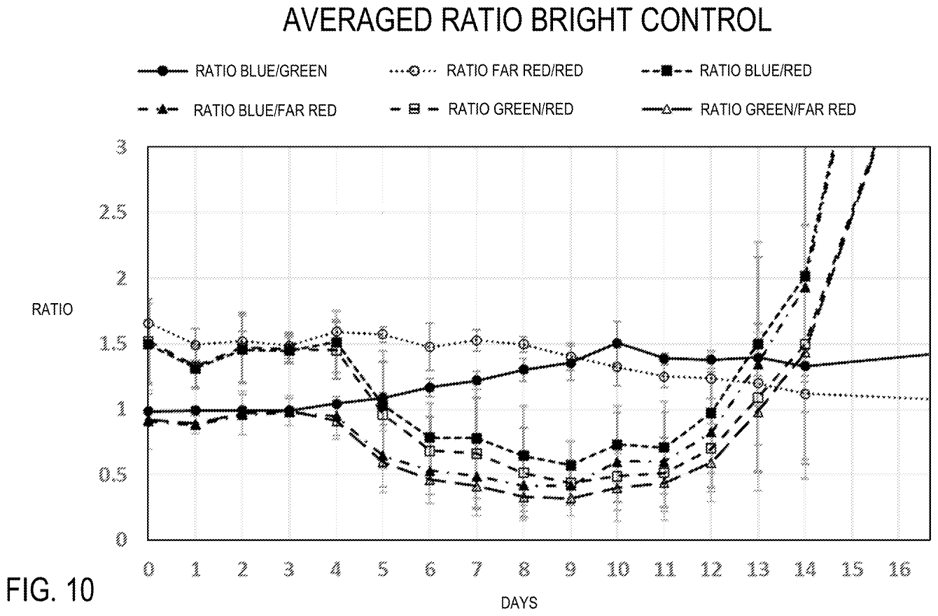

FIG. 10 illustrates average ratios of different bandwidths of detected fluorescence over time for the stressed plants. As illustrated, the Blue to Red, the Blue to Far Red, the Green to Red, and the Green to Far-Red ratios significantly decrease and the Blue to Green ratio increases starting at day 4, which signifies the detection of a drought condition. The change in ratio is significantly more pronounced than for the change in the Red to Far Red ratio making it easier to detect. The stress detection using Blue and or Green Fluorescence is also significantly faster: stress can be detected after 4 days in comparison to the 8 days it takes for Red and Far-Red detection. In comparison, image analysis also takes about 8 days to detect.

FIG. 11 is a schematic diagram depicting an example detection module 1100 that includes a set of detectors 1112. Detection module 1100 is an example of detection module 152 of FIG. 1. The set of detectors 1112 is an example of the set of detectors 112 of FIG. 1. In this example, detectors 1122-1, 1122-2, 1122-N of the set of detectors 1112 take the form of transimpedance amplifier photodiodes. However, other suitable detectors can be used.

Detection module 1100 includes a set of optical filters 1130. At least a portion of light 1101 received by detection module 1100 passes through the set of optical filters 1130. In the example of FIG. 11, the set of filters 1130 includes one or more longpass filters 1132, and each detector of the set of detectors 1112 includes a respective optical filter 1134-1, 1134-2, 1134-N. Each of optical filters 1134-1, 1134-2, 1134-N can be configured to pass light within a respective wavelength range that differs among the set of detectors 1112 such that each detector receives light of a different wavelength range. Longpass filters 1132 can be configured to pass light within a broader wavelength range that includes the wavelength ranges of optical filters 1134-1, 1134-2, 1134-N.

Detection module 1100 can be operatively coupled to a power source 1102. Power source 1102 can take the form of an electrical outlet of an electrical utility or a battery, as examples. As another example, power source 1102 can form part of detection module 1100 or controller 110 of FIG. 1, as a battery. Detection module 1100 can communicate with various components of controller 110, as depicted schematically at 1104 in FIG. 11. Such communications can include bidirectional electronic signals.

Detection module 1100 includes a multiplexer 1140. Electrical signals generated by each detector of the set of detectors 1112 responsive to and based on light received by that detector is received by multiplexer 1140, which multiplexes the electrical signals to obtain a multiplexed signal that is output by the multiplexer. Detection module 1100 includes a signal filter and isolation stage 1142 that can process the multiplexed signal received from multiplexer 1140 through signal filtering, and can further provide electrical isolation between the set of detectors 1112 and downstream circuitry.

Detection module 1100 includes a set of signal amplifier stages 1144 and sample and hold circuitry 1146. Electrical signals output by signal filter and signal isolation stage 1142 is provided to the positive side of a first signal amplifier stage 1148-1 of the set of signal amplifier stages 1144, and to sample and hold circuity 1146. A negative side of first signal amplifier stage 1148-1 is electrically coupled to an output of sample and hold circuitry 1146. An output of first signal amplifier stage 1148-1 can be passed to one or more additional signal amplifier stages for further amplification of the signal, an example of which include a second signal amplifier stage 1148-2. The set of signal amplifier stages 1144 outputs the amplified signal as a signal 1150 to controller 110 of FIG. 1. In other examples, some or all of the electronic components of detection module 1100, including multiplexer 1140, signal filter and signal isolation stage 1142, sample and hold circuitry 1146, and the set of signal amplifier stages 1144 can form part of controller 110 of FIG. 1.

The detection modules disclosed herein are configured to detect the fluorescence emitted by the plants in response to the excitation signal and converts the optical response to an electrical signal. In some embodiments the Detector module comprises one or more individual photodiodes. In one embodiment the photodiodes may be silicon based.

In one embodiment optical bandpass filters, such as described with reference to FIG. 11, may be placed on the photodiodes to limit the wavelength range of the detected fluorescence. It is understood that the bandpass filter can be integrated into the photodiode itself. The number of bandpass filters, the overall measurement range, the center wavelengths of the filters and the width of the filters may be selected in accordance with the fluorescence spectrum to be observed. In some embodiments the wavelength range to be observed is between 400 nm and 780 nm with four photodiodes with filters centered around 480 nm, 530 nm, 685 nm and 740 nm. The width of the filter may be wide such as 50 nm Full Width Half Maximum (FWHM), or as narrow as 10 nm or 20 nm in order to avoid crosstalk between channels and the excitation source. For example, it may be of value to have one filter that passes light from 450-500 nm, a second filter from 500-550 nm, a third filter from 670-700 nm, and a fourth filter from 700-750 nm.

The detection of Blue and or Green fluorescence is of particular interest as the ratio of Blue to Green fluorescence and the ratio of Blue or Green to Red or Far Red fluorescence can be a suitable indicator for plant health/stress.

In one embodiment the Detector module comprises only two photodiodes. A photodiode with a filter centered around 480 nm and one with a filter centered around 530 nm wavelengths in order to monitor the Blue to Green fluorescence ratio. In a different embodiment the two photodiodes may be centered around Blue and Red or Blue and Far Red or Green and Red or Green and Far Red to observe the ratio of the respective fluorescent responses. In yet another embodiment the detector may include three photodiodes. It is also understood that more than four photodiodes could be used. The addition of further photodiodes will increase the spectral resolution of the measured signal and may be of benefit in analyzing plant health.

In another embodiments the detection module may comprise a fully integrated photodiode system as is offered for example by AMS-Osram or one or more phototransistors, a spectrometer, a linear CCD array or other means to detect the fluorescence signal emitted by the plants.

As described with reference to FIG. 11, the detection module can include electronic components to convert the fluorescence emission of the plant into an electrical signal. The detection module may comprise transimpedance amplifiers, single stage or dual stage operational amplifiers, instrumentation amplifiers or suitable other components to detect the fluorescence emission and generate and condition an electrical signal. The detection module may also comprise multiplexers to keep the count of operational amplifiers and or instrumentation amplifiers and the cost of the system low.

FIG. 12 is a schematic diagram depicting an example excitation module 1200 that includes a set of emitters 1214. Excitation module 1200 is an example of excitation module 154 of FIG. 1. The set of emitters 1214 is an example of the set of emitters 114 of FIG. 1. In this example, emitters 1224-1, 1224-2, 1224-M of the set of emitters 1214 take the form of LEDs. Furthermore, in the example of FIG. 12, excitation module 1200 includes a switch 1210 operatively coupled to a power source 1230. Alternatively, switch 1210 can form part of controller 110 of FIG. 1. In the example of FIG. 12, power source 1230 is located off-board excitation module 1200. For example, power source 1230 can take the form of an electrical output of an electrical utility or can take the form of a battery. In another example, power source 1230 can form part of controller 110 of FIG. 1, and can take the form of a battery. In another example, power source 1230 can form part of excitation module 1200, and can take the form of a battery. As depicted schematically at 1232, switch 1210 is operable between an on state and an off state in response to a signal from controller 110 of FIG. 1. In the on state, each emitter of the set of emitters 1214 emits light 1234, as depicted schematically in FIG. 12. In the example of FIG. 12, the set of emitters 1214 are operatively coupled to ground 1236 on an opposite side of power source 1230.

In some examples, controller 110 of the measurement system may perform processing of measurement data, including data analysis such as averaging and background subtraction. The controller can also write the gathered data to a local server or cloud storage. The plant health may be sampled every 15 minutes, as an example.

The photon flux density of the UV LEDs may be 25-150 μmol/m2/s centered around 365 nm at 500 mm distance. An optical short wavelength pass filter with cut off around 400 nm may be used to remove significantly all emission above 400 nm from the excitation light. The frequency of the pulse may be 30 Hz with a 50% duty cycle and the fluorescence measurement may be performed in 100-1000 microsecond intervals. A measurement delay of 100-1000 microsecond may be chosen to allow for stabilization of the excitation source or fluorescence signal. The individual measurements are written into an array for further/subsequent subtraction of noise in the controller or in the application software and/or the measurements are averaged. The measurement is performed in the on cycle and in the off cycle of the pulse. The off cycle provides information about the environment and background signal and may be subtracted and or also stored to the cloud for further analysis. The measurements may be performed for one cycle (on/off), several cycles or a large number of cycles depending on the application and the noise level. Measurements for the individual cycles may be stored to the cloud for further analysis or averaged for improved signal quality. The Detector module may comprise 4 photodiodes with filters, one with a filter centered around 480 nm, one around 530 nm, one around 680 nm and one around 740 nm. The measurement data may be written to a local server/computer via wired or wireless protocol. The application software will monitor the measurements of a multitude of systems, record the health status of the plant over time and alert the user to detected stress. A fifth dark photodiode may be used to subtract background noise on the PCB. In addition, a long pass window may be placed over the photodiodes in order to remove significantly all excitation light from the light incident on the photodiodes. The Excitation module may comprise an optic to provide uniform photon flux at 500 mm distance of approximately 300 mm diameter. The Detector module may comprise an optic with and FOV to match the area illuminated by the excitation module.

In yet a different embodiment the system comprises a fully integrated multi spectral sensor such as the AMS7343. The AMS7343 sensor comprises a monolithic photodiode array integrated with optical filters providing access to 12 different spectral regions from 400 to 850 nm. The AMS7343 also comprises ADCs and a serial I2C interface, The system also comprises the Excitation module similar as in the previous descriptions and a Control module that sends control signals to the Excitation module and Controls the measurements of the AMS7343 sensor. The Control module also reads out the spectral information from the AMS7343 sensor. As the sensitivity of the AMS module is lower than that of individual photodiodes the measurement sequence will be slower and will need to be adjusted. The duration of the pulse train may be 100 ms to 1 min. The individual on pulse and off pulse of the excitation source may be 10 ms to 10 s. The measurement time (detector integration time) itself may be 10 ms to 10 s both for the on pulse and off pulse. Several measurements may be taken to improve signal quality and either all measurements or the average of the measurements may be written to the local server or cloud.

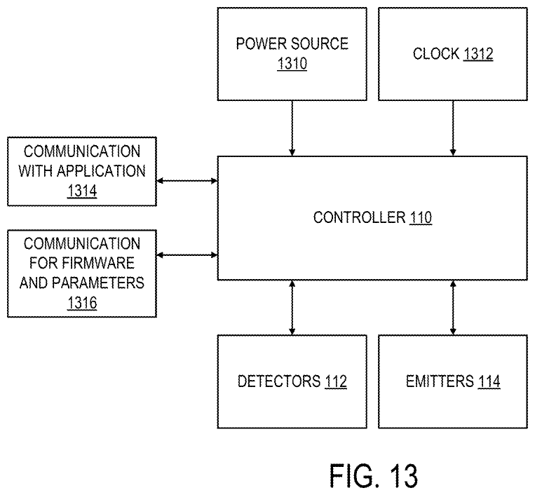

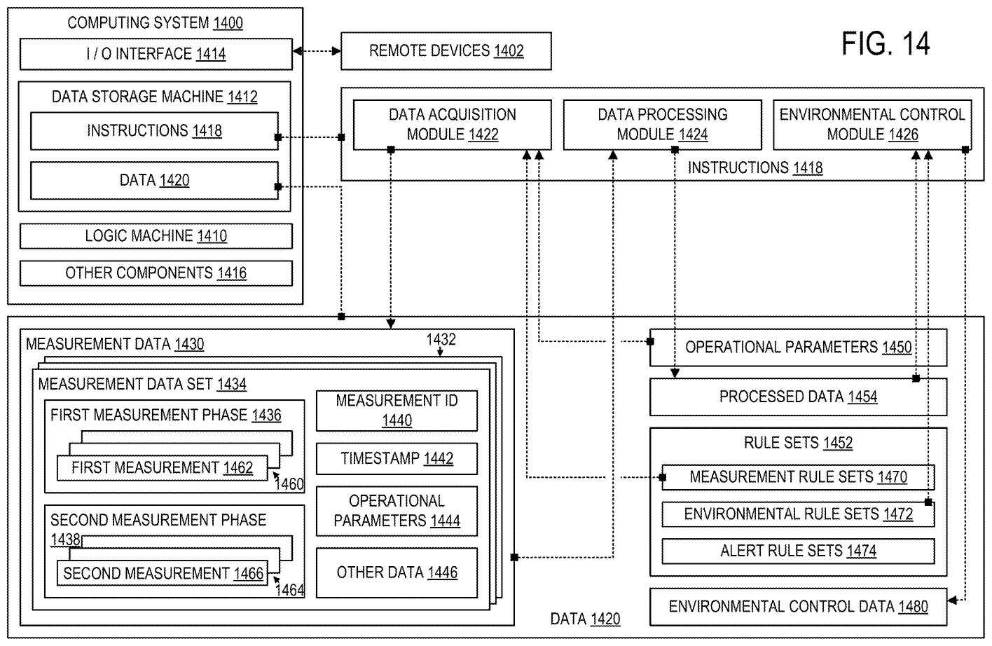

FIG. 13 is a schematic diagram depicting additional aspects of controller 110 of FIG. 1. Controller 110 is an example of a computing system or computing device. In at least some examples, controller 110 can take the form of a microcontroller that includes an integrated logic machine and data storage machine having instructions stored thereon that are executable by the logic machine. Additional aspects of a computing system and computing device that includes a logic machine and a data storage machine are described in further detail with reference to FIG. 14.

In FIG. 13, controller 110 communicates with the set of detectors 112 and the set of emitters 114 as previously described with reference to FIG. 1. Additionally, within FIG. 13, controller receives electrical power from a power source 1310, which can include an electrical output of an electrical utility or a battery, as examples. In another example, power source 1310 can form part of controller 110, and can take the form of a battery. Controller 110 can receive a clock signal from a clock 1312, depicted schematically in FIG. 13 to enable controller 110 to enact the methods and operations described herein according to a particular timing. Clock 1312 can form part of controller 110 in at least some examples.

Controller 110 can communicate with various other devices as previously described with reference to FIGS. 1 and 2. At 1314, FIG. 13 schematically depicts controller 110 communicating with an application program of another device. The application program can refer to one or more of on-premises program 214, server program 224, and/or mobile program 234 of FIG. 2. At 1316, FIG. 13 schematically depicts controller 110 communicating with another device to receive firmware or firmware updates, and to receive various operational parameters that define how measurements are to be conducted via detectors 112 and emitters 114. Communications 1316 can be received by controller 110 from one or more of on-premises computing device 210, remote server system 220, and/or mobile computing device 230 of FIG. 2. The various communications depicted in FIG. 13 are examples of control input 140 and output data 142 of FIG. 1.

The methods and operations described herein may be tied to a computing system of one or more computing devices. In particular, such methods and operations may be implemented as a computer-application program or service, an application-programming interface (API), a library, and/or other computer-program product.

FIG. 14 is a schematic diagram of a computing system 1400 that can perform the methods and operations described herein. Computing system 200 of FIG. 2 is an example of computing system 1400. Accordingly, computing system 1400 can refer to any combination of measurement system 100, controller 110, on-premises computing device 210, remove server system 220, mobile computing device 230, environmental control system 240, and/or controllers 242 of FIG. 2.

Computing system 1400 includes a logic machine 1410, a storage machine 1412, an input/output (IO) subsystem 1414 by which the computing system can communication with remote device 1402, and other suitable components 1416. It will be understood that computing system 1400 is schematically depicted in FIG. 14 in simplified form. Computing system 1400 may take the form of one or more personal computers, server computers, network computing devices, mobile computing devices, and/or other computing devices.

Logic machine 1410 includes one or more physical logic devices configured to execute instructions 1418 and/or other data 1420 stored in data storage machine 1412. For example, the logic machine may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

In FIG. 14, instructions 1418 include a data acquisition module 1422, a data processing module 1424, and an environmental control module 1426, as an example. Instructions 1418 include or take the form of on-premises program 214, server program 224, and mobile program 234 of FIG. 2. Additionally, instructions 1418 can include or take the form of instructions executable at or by controller 110 of FIG. 1 and/or instructions executable at or by controllers 242 of FIG. 2. Data storage machine 1412 can include or take the form of on-premises data storage 212, remote data storage 222, and mobile data storage 232. Additionally, data storage machine 1412 can include or take the form of a data storage machine of controller 110 of FIG. 1 and/or controllers 242 of FIG. 2.

Logic machine 1410 may include one or more processors configured to execute software instructions. Additionally or alternatively, the logic machine may include one or more hardware or firmware logic machines configured to execute hardware or firmware instructions. Processors of the logic machine may be single-core or multi-core, and the instructions executed thereon may be configured for sequential, parallel, and/or distributed processing. Individual components of the logic machine optionally may be distributed among two or more separate devices, which may be remotely located and/or configured for coordinated processing. Aspects of the logic machine may be virtualized and executed by remotely accessible, networked computing devices configured in a cloud-computing configuration.

Data storage machine 1412 includes one or more physical storage devices configured to hold instructions 1418 and other data 1420 executable by logic machine 1410 to perform or otherwise implement the methods and operations described herein. When such methods and operations are performed or implemented, the state of data storage machine 1412 may be transformed-e.g., to hold different data.

Data storage machine 1412 may include removable and/or built-in devices. Data storage machine 1412 may include optical memory, semiconductor memory (e.g., RAM, EPROM, EEPROM, etc.), and/or magnetic memory (e.g., hard-disk drive, floppy-disk drive, tape drive, MRAM, etc.), among others. Data storage machine 1412 may include volatile, nonvolatile, dynamic, static, read/write, read-only, random-access, sequential-access, location-addressable, file-addressable, and/or content-addressable devices.

It will be appreciated that data storage machine 1412 includes one or more physical storage devices. However, aspects of the instructions (e.g., instructions 1418) and other data described herein alternatively may be propagated by a communication medium (e.g., an electromagnetic signal, an optical signal, etc.) that is not held by a physical device for a finite duration.

Aspects of logic machine 1412 and storage machine 1410 may be integrated together into one or more hardware-logic components. Such hardware-logic components may include field-programmable gate arrays (FPGAs), program-and application-specific integrated circuits (PASIC/ASICs), program-and application-specific standard products (PSSP/ASSPs), system-on-a-chip (SOC), and complex programmable logic devices (CPLDs), for example.

The terms “module,” “program,” and “engine” may be used to describe an aspect of computing system 1400 implemented to perform a particular function. In some cases, a module, program, or engine may be instantiated via logic machine 1410 executing instructions 1418 and/or other data 1420 held by storage machine 1412. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

Data acquisition module 1422 can be implemented by computing system 1400 to obtain measurement data 1430 by conducting one or more measurement operations based on operational parameters 1450 and measurement rule sets 1470. Operational parameters 1450 can define aspects of the measurement operations conducted to obtain measurement data 1430, including how measurement operations are to be conducted. Examples of operational parameters 1450 include emission wavelength ranges of excitation light to emitted by emitters, the quantity, duration, timing, and intensity of excitation light pulses to be emitted by the emitters, observation wavelength ranges for light to be measured by detectors, and the quantity, duration, and timing of measurements to be obtained via the detectors. Measurement rule sets 1470 can include a schedule that defines when and where measurement operations are to be conducted.

Measurement data 1430 includes a plurality of measurement data sets 1432, an example of which includes measurement data set 1434. Each measurement data set corresponds to a measurement operation, and includes measurement data for a first measurement phase 1436 and a second measurement phase 1438. First measurement phase 1436 includes measurement data captured during a period that is configured to measure a baseline of fluorescent light. First measurement phase 1436 includes a first set of measurements 1460, of which first measurement 1462 is an example. Second measurement phase 1438 includes measurement data captured during a period that is configured to measure fluorescence response of a subject plant to excitation light. Second measurement phase 1438 includes a second set of measurements 1464 of which second measurement 1466 is an example.

Each measurement data set includes a measurement identifier 1440 that uniquely identifies and distinguishes the measurement data set corresponding to a particular measurement operation from other measurement data sets corresponding to other measurement operations. Measurement identifier 1440 can identify and/or be associated with other data that identifies the test subject (e.g., a particular plant or group of plants), a location of the test subject, the measurement system, emitters, and detectors used to conduct measurements of fluorescence response, and other suitable information.

Each measurement data set includes a timestamp 1442 that identifies a time or time range (e.g., date and clock time) that the measurement operation was conducted and the measurements of the measurement data set where obtained.

Each measurement data set includes or otherwise identifies operational parameters 1444 that were used or otherwise implemented by the measurement system to conduct the measurement operation. Operational parameters 1444 can refer to a subset of operational parameters 1450. Each measurement data set may include other data 1446 that identifies aspects of the measurement operation and/or measurements obtained by performing that measurement operation.

Data processing module 1424 is configured to obtain measurement data 1430 and process that data to obtain processed data 1454. Processed data 1454 for a particular measurement data set 1434 can be associated with measurement ID 1440, timestamp 1442, operational parameters 1444, and other data 1446 to enable the processed data obtained from a measurement operation from other processed data obtained from other measurement operations.

In addition to measurement rule sets 1470, rule sets 1452 can include environmental rule sets 1472 that define how environmental operating conditions are to be varied in response to measurement data 1430 and/or processed data 1454; and alert rule sets 1474 that can define criteria for when and how alerts are to be generated and output by the computing system in response to measurement data 1430 and/or processed data 1454.

Environmental control module 1426 is configured to obtain processed data 1454 and environmental rule sets 1472, and to enact control strategies with respect to environmental conditions of the environment from which the measurement data 1430 was obtained.

The foregoing description of examples has been presented for purposes of illustration and description. It is not intended to be exhaustive or limiting to the forms described. Numerous modifications are possible in light of the above teachings. Some of those modifications have been discussed, and others will be understood by those skilled in the art. The examples were chosen and described in order to illustrate principles of various examples as are suited to particular uses contemplated. The scope is, of course, not limited to the examples set forth herein, but can be employed in any number of applications and equivalent devices by those of ordinary skill in the art.

In the accompanying drawings, structures are illustrated that, together with the detailed description provided below, describe example embodiments of the disclosed apparatus and methods. Where appropriate, like elements are identified with the same or similar reference numerals. Elements shown as a single component can be replaced with multiple components. Elements shown as multiple components can be replaced with a single component. The drawings may not be to scale. The proportion of certain elements may be exaggerated for the purpose of illustration.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A method of monitoring plant status performed by a computing system of one or more computing devices, the method comprising:
performing a plurality of measurement operations periodically over a period of time in which each measurement operation includes:
emitting, via one or more emitters, excitation light within an emission wavelength range into an environment containing one or more subject plants,
prior to emitting the excitation light, obtaining via one or more detectors having a field of view containing the one or more subject plants, at least a first measurement of light intensity within a first observation wavelength range that differs from the emission wavelength range, and at least a first measurement of light intensity within a second observation wavelength range that differs from the emission wavelength range and the first observation wavelength range,
during and/or after emission of the excitation light, obtaining via the one or more detectors, at least a second measurement of light intensity within the first observation wavelength range, and at least a second measurement of light intensity within the second observation wavelength range; and
for each measurement operation performed:
subtracting the first measurement of light intensity from the second measurement of light intensity within the first observation wavelength range to obtain a first measurement of fluorescence response of the one or more plants within the first observation wavelength range,
subtracting the first measurement of light intensity from the second measurement of light intensity within the second observation wavelength range to obtain a second measurement of fluorescence response of the one or more plants within the second observation wavelength range, and
comparing the first measurement of fluorescence response to the second measurement of fluorescence response to obtain a result indicative of the plant status; and
outputting the result indicative of the plant status for each measurement operation.

2. The method of claim 1, further comprising:
outputting an alert responsive to any of the results exceeding a threshold value.

3. The method of claim 1, further comprising:
outputting a control signal to one or more physical actuators to control an operating condition of the environment containing the one or more plants responsive to any of the results exceeding a threshold value.

4. The method of claim 1, further comprising:
identifying a rate of change of the results over the period of time;
comparing the rate of change to a threshold rate of change; and
outputting an alert responsive to the rate of change exceeding the threshold rate of change.

5. The method of claim 1, further comprising:
identifying a rate of change of the results over the period of time;
comparing the rate of change to a threshold rate of change; and
outputting a control signal to one or more physical actuators to control an operating condition of the environment containing the one or more plants responsive to the rate of change exceeding the threshold rate of change.

6. The method of claim 1, wherein the emission wavelength range is less than the first observation wavelength range and the second observation wavelength range.

7. The method of claim 6, wherein the emission wavelength range includes ultraviolet light;
wherein the first observation wavelength range includes red light and/or near red light; and
wherein the second observation wavelength range includes green light and/or blue light.

8. The method of claim 1, wherein the first measurement of light intensity within the first observation wavelength range and the first measurement of light intensity within the second observation wavelength range are captured in parallel via the one or more detectors during the same sample period; and
wherein the second measurement of light intensity within the first observation wavelength range and the second measurement of light intensity within the second observation wavelength range are captured in parallel via the one or more detectors during the same sample period.

9. The method of claim 1, wherein the first measurement of light intensity within the first observation wavelength range and the first measurement of light intensity within the second observation wavelength range are captured in sequence via the one or more detectors during different sample periods; and
wherein the second measurement of light intensity within the first observation wavelength range and the second measurement of light intensity within the second observation wavelength range are captured in sequence via the one or more detectors during different sample periods.

10. The method of claim 1, wherein the first measurement of light intensity within the first observation wavelength range is one of a first set of multiple measurements within the first observation wavelength range that are obtained for each measurement operation;
wherein the first measurement of light intensity within the second observation wavelength range is one of a first set of multiple measurements within the second observation wavelength range that are obtained for each measurement operation;
wherein the second measurement of light intensity within the first observation wavelength range is one of a second set of multiple measurements within the first observation wavelength range that are obtained for each measurement operation;
wherein the second measurement of light intensity within the second observation wavelength range is one of a second set of multiple measurements within the second observation wavelength range that are obtained for each measurement operation;
wherein the first measurement of fluorescence response is obtained by subtracting the first set of measurements of light intensity within the first observation wavelength range from the second set of measurements of light intensity within the first observation wavelength range; and
wherein the second measurement of fluorescence response is obtained by subtracting the first set of measurements of light intensity within the second observation wavelength range from the second of measurements of light intensity within the second observation wavelength range.

11. A computing system for monitoring plant status, the computing system comprising:
a logic machine; and
a data storage machine having instructions stored thereon executable by the logic machine to:
perform a plurality of measurement operations periodically over a period of time in which each measurement operation includes:
emit, via one or more emitters, excitation light within an emission wavelength range into an environment containing one or more subject plants,
prior to emitting the excitation light, obtain via one or more detectors having a field of view containing the one or more subject plants, at least a first measurement of light intensity within a first observation wavelength range that differs from the emission wavelength range, and at least a first measurement of light intensity within a second observation wavelength range that differs from the emission wavelength range and the first observation wavelength range,
during and/or after emission of the excitation light, obtain via the one or more detectors, at least a second measurement of light intensity within the first observation wavelength range, and at least a second measurement of light intensity within the second observation wavelength range; and
for each measurement operation performed:
subtract the first measurement of light intensity from the second measurement of light intensity within the first observation wavelength range to obtain a first measurement of fluorescence response of the one or more plants within the first observation wavelength range,
subtract the first measurement of light intensity from the second measurement of light intensity within the second observation wavelength range to obtain a second measurement of fluorescence response of the one or more plants within the second observation wavelength range, and
compare the first measurement of fluorescence response to the second measurement of fluorescence response to obtain a result indicative of the plant status; and
output the result indicative of the plant status for each measurement operation.

12. A device, comprising:
a data storage machine having instructions stored thereon executable by a logic machine to:
perform a plurality of measurement operations periodically over a period of time in which each measurement operation includes:
emit, via one or more emitters, excitation light within an emission wavelength range into an environment containing one or more subject plants,
prior to emitting the excitation light, obtain via one or more detectors having a field of view containing the one or more subject plants, at least a first measurement of light intensity within a first observation wavelength range that differs from the emission wavelength range, and at least a first measurement of light intensity within a second observation wavelength range that differs from the emission wavelength range and the first observation wavelength range,
during and/or after emission of the excitation light, obtain via the one or more detectors, at least a second measurement of light intensity within the first observation wavelength range, and at least a second measurement of light intensity within the second observation wavelength range; and
for each measurement operation performed:
subtract the first measurement of light intensity from the second measurement of light intensity within the first observation wavelength range to obtain a first measurement of fluorescence response of the one or more plants within the first observation wavelength range,
subtract the first measurement of light intensity from the second measurement of light intensity within the second observation wavelength range to obtain a second measurement of fluorescence response of the one or more plants within the second observation wavelength range, and
compare the first measurement of fluorescence response to the second measurement of fluorescence response to obtain a result indicative of the plant status; and
output the result indicative of the plant status for each measurement operation.
